(19) United States
(12) Patent Application Publication
(10) Pub. No.: US 2016/0168933 A1
(43) Pub. Date: Jun. 16, 2016

(54) INTELLIGENT SENSOR SYSTEMS AND METHODS

(71) Applicant: Mathena, Inc., El Reno, OK (US)

(72) Inventors: Levent Aktas, Norman, OK (US); Timothy Long, Oklahoma City, OK (US); Matthew B. Green, Norman, OK (US); John Mathena, Edmond, OK (US)

(21) Appl. No.: 14/963,839

(22) Filed: Dec. 9, 2015

Related U.S. Application Data
(60) Provisional application No. 62/089,913, filed on Dec. 10, 2014.
(60) Provisional application No. 62/173,633, filed on Jun. 10, 2015.

(51) Int. Cl. E21B 21/01; E21B 21/00; G01N 33/28

(52) U.S. Cl. CPC E21B 21/01; G01N 33/2823; E21B 21/002

(57) ABSTRACT

According to one aspect, an intelligent sensor system is adapted to monitor at least a first operating parameter of a first vessel during oil and gas exploration and production operations. The system includes a first sensor housing assembly, which includes a first sensor adapted to measure a first physical property associated with the first vessel. The monitored first operating parameter is, or is based on, the first physical property measured by the first sensor. A control unit may be in communication with the first sensor. The control unit may be adapted to be in communication with an electronic drilling recorder (EDR). According to another aspect, a system is located at a drilling rig site, and includes first and second sensor housing assemblies connected to first and second vessels, respectively. According to yet another aspect, an intelligent sensor system is adapted to monitor an operating parameter of a gas vent line.

### CROSS-REFERENCE TO RELATED APPLICATIONS

This application claims the benefit of the filing date of, and priority to, U.S. patent application No. 62/089,913, filed Dec. 10, 2014, the entire disclosure of which is hereby incorporated herein by reference.

This application claims the benefit of the filing date of, and priority to, U.S. patent application No. 62/173,633, filed Jun. 10, 2015, the entire disclosure of which is hereby incorporated herein by reference.

This application is related to the following applications: U.S. application Ser. No. 13/000,964, filed Jun. 30, 2008, now U.S. Pat. No. 8,641,811, issued Feb. 4, 2014; and U.S. application Ser. No. 14/049,726, filed Oct. 9, 2013, now U.S. Pat. No. 8,784,545, issued Jul. 22, 2014, the entire disclosures of which are hereby incorporated herein by reference.

### TECHNICAL FIELD

This disclosure relates in general to sensor systems and, in particular, to an intelligent sensor system for monitoring one or more operating parameters of either a vessel or a vent line and, in several exemplary embodiments, controlling aspects associated with the operation of the vessel or vent line.

### BACKGROUND OF THE DISCLOSURE

During the drilling of an oil or gas well, different materials may be discharged from the well. The discharged materials may include mixtures of solid, liquid, and gas materials. The discharged materials may be flammable. The discharged materials may be conveyed through different vessels and gas vent lines, which are located at the drilling rig site. Examples of such vessels may include mud-gas separator vessels, shale-gas separator vessels, mud-containment vessels, or any combination thereof. In many cases, one or more operating parameters associated with the different vessels and gas vent lines are not able to be intelligently monitored, remotely or otherwise. Moreover, aspects associated with the operation of one or more of the vessels and gas vent lines are not able to be sufficiently controlled, remotely or otherwise. Therefore, what is needed is a system, method, kit, apparatus, or assembly that addresses one or more of these issues, and/or other issue(s).

### SUMMARY

In a first aspect, there is provided a system adapted to monitor at least a first operating parameter of a first vessel, the first vessel defining a first internal region. The system includes a first sensor housing assembly, the first sensor housing assembly including: a first fitting adapted to be connected to the first vessel, the first fitting defining a first internal passage adapted to be in fluid communication with the first internal region; a second fitting adapted to be connected to the first vessel, the second fitting defining a second internal passage adapted to be in fluid communication with the first internal region; a housing extending between the first and second fittings, the housing defining a second internal region adapted to be in fluid communication with the first internal region via the first and second passages; and a first sensor connected to at least one of the first fitting, the second fitting, and the housing. The first sensor is adapted to measure a first physical property associated with the first vessel. The monitored first operating parameter is, or is based on, the first physical property measured by the first sensor.

In an exemplary embodiment, system further includes a control unit adapted to be in communication with the first sensor and adapted to receive from the first sensor first measurement data associated with the first physical property; wherein the control unit is adapted to determine the first operating parameter based on the first measurement data.

In another exemplary embodiment, the control unit is adapted to be in communication with an electronic drilling recorder (EDR). The control unit is adapted to send to the EDR first parameter data associated with first operating parameter.

In yet another exemplary embodiment, the first physical property is a fluid level within the first vessel; the first sensor is a level sensor adapted to measure the fluid level within the first vessel; the level sensor is one of a guided wave level sensor and a non-contact radar level sensor; the first sensor housing assembly further includes a port in fluid communication with the second internal region of the housing; and the level sensor is positioned, relative to the port, so that the level sensor can measure the fluid level within the first vessel.

In certain exemplary embodiments, the housing defines a longitudinally-extending center axis; wherein the first housing assembly further includes a cap lying in a plane that is perpendicular to the center axis of the housing; wherein the first port is formed through the cap and the level sensor is connected to the cap; and wherein the perpendicular orientation between the center axis and the plane in which the cap lies facilitates the measurement of the fluid level by the level sensor.

In an exemplary embodiment, the level sensor is the guided wave level sensor, the guided wave level sensor including a probe extending through the port and within the second internal region of the housing.

In another exemplary embodiment, the level sensor is the non-contact radar level sensor, at least a portion of which is positioned adjacent the port.

In yet another exemplary embodiment, the housing is a tubular housing; wherein each of the first and second fittings is connected directly to the tubular housing; and wherein the respective direct connections between the tubular housing and each of the first and second fitting are weld-less, within the second internal region defined by the tubular housing, increasing smoothness along respective internal surfaces of the tubular housing and the first and second fittings, facilitates the measurement of the fluid level by the non-contact radar level sensor.

In still yet another exemplary embodiment, the system includes a flange directly connected to an end of the tubular housing, wherein the cap is connected to the flange.

In certain exemplary embodiments, the housing is a tubular housing, the tubular housing including opposing first and second end portions; and wherein the system further includes: a first t-fitting connected to the first end portion of the tubular housing, wherein the first fitting is a part of the first t-fitting; and a second t-fitting connected to the second end portion of the tubular housing, wherein the second fitting is part of the second t-fitting.

In an exemplary embodiment, the first sensor housing assembly further includes a second sensor connected to at least one of the first fitting, the second fitting, and the housing; and wherein the second sensor is adapted to measure a second physical property associated with the first vessel.

In another exemplary embodiment, the first sensor housing assembly further includes: a first end portion at which the first fitting is located; a second end portion at which the second fitting is located, the second end portion opposing the first end portion; a first port formed at the first end portion of the first sensor housing assembly, wherein the first port is in fluid communication with the second internal region of the housing; and a second port formed at the second end portion of the first sensor housing assembly, wherein the second port is in fluid communication with the second internal region of the housing; wherein the first and second sensors are first and second pressure sensors, respectively; and wherein the first and second pressure sensors are positioned adjacent the first and second ports, respectively.

In yet another exemplary embodiment, the first physical property adapted to be measured by the first pressure sensor is mud column pressure within the first vessel; and wherein the second physical property adapted to be measured by the second pressure sensor is gas vessel pressure within the first vessel.

In still yet another exemplary embodiment, the monitored first operating parameter is mud density.

In certain exemplary embodiments, mud is adapted to be discharged from the first vessel via a discharge valve, the discharge valve having operating characteristics; and wherein the monitored first operating parameter is mud discharge flow rate, the mud discharge flow rate being based on at least the mud column pressure and the operating characteristics of the discharge valve.

In an exemplary embodiment, the first physical property to be measured by the first pressure sensor is pressure at a lower end portion of the first vessel; and wherein the second physical property to be measured by the second pressure sensor is pressure at the upper end portion of the first vessel.

In another exemplary embodiment, the monitored first operating parameter is selected from the group consisting of: a fluid level within the first vessel; an operating pressure within the first vessel; and liquid density within the first vessel.

In yet another exemplary embodiment, the system includes a control unit adapted to be in communication with the first sensor and adapted to receive from the first sensor first measurement data associated with the first physical property; wherein the control unit is adapted to determine the first operating parameter based on the first measurement data; and wherein mud is adapted to be discharged from the first vessel via a discharge valve; and wherein the control unit is adapted to automatically control the discharge valve based on the first operating parameter.

In still yet another exemplary embodiment, the first vessel is selected from the group consisting of a mud-gas separator vessel; a shale-gas separator vessel; and a mud-gas containment vessel.

In certain exemplary embodiments, the system includes a second sensor housing assembly, the second sensor housing assembly including a second sensor adapted to measure a second physical property associated with a second vessel; and a control unit adapted to be in communication with each of the first and second sensors; wherein the control unit is adapted to receive from the first sensor first measurement data associated with the first physical property; wherein the control unit is adapted to receive from the second sensor second measurement data associated with the second physical property; wherein the control unit is adapted to determine the first operating parameter based on the first measurement data; wherein the control unit is adapted to determine a second operating parameter of the second vessel based on the second measurement data; and wherein the second operating parameter is, or is based on, the second physical property measured by the second sensor.

In an exemplary embodiment, the first and second vessels are located at a drilling ring site; and wherein each of the first and second vessels is selected from the group consisting of: a mud-gas separator vessel; a shale-gas separator vessel; and a mud-gas containment vessel.

In another exemplary embodiment, the system includes the first vessel, wherein the first vessel is a mud-gas separator vessel; the second vessel, wherein the second vessel is a mud-gas containment vessel; a gas vent line via which the mud-gas containment vessel is in fluid communication with the mud-gas separator vessel; wherein the first sensor housing assembly is connected to the mud-gas separator vessel; wherein the second sensor housing assembly is connected to the mud-gas containment vessel; wherein the first and second sensors are level sensors adapted to measure respective fluid levels within the mud-gas separator vessel and the mud-gas containment vessel; and wherein the monitored first operating parameter of the mud-gas separator vessel provides an early warning of potential flooding within the mud-gas separator vessel and an even earlier warning of potential flooding within the mud-gas containment vessel.

In a second aspect, there is provided a monitoring system located at a drilling rig site, the system including a first vessel; a second vessel in fluid communication with the first vessel; a first sensor housing assembly connected to the first vessel, the first sensor housing including a first sensor adapted to measure a first physical property associated with the first vessel; a second sensor housing assembly connected to the second vessel, the second sensor housing including a second sensor adapted to measure a second physical property associated with the second vessel; and a control unit adapted to be in communication with each of the first and second sensors to determine and monitor first and second operating parameters of the first and second vessels, respectively; wherein each of the first and second operating parameters is, or is based on, the first and second physical properties, respectively.

In an exemplary embodiment, the system includes an electronic drilling recorder (EDR) in communication with the control unit; wherein the control unit is adapted to send to the EDR parameter data associated with first and second operating parameters.

In another exemplary embodiment, each of the first and second vessels is selected from the group consisting of: a mud-gas separator vessel; a shale-gas separator vessel; and a mud-gas containment vessel.

In yet another exemplary embodiment, the first vessel is a mud-gas separator vessel; wherein the second vessel is a mud-gas containment vessel; wherein the first sensor housing assembly is connected to the mud-gas separator vessel; wherein the second sensor housing assembly is connected to the mud-gas containment vessel; wherein the first and second sensors are level sensors adapted to measure respective fluid levels within the mud-gas separator vessel and the mud-gas containment vessel; and wherein the monitored first operating parameter of the mud-gas separator vessel provides an early warning of potential flooding within the mud-gas separator vessel and an even earlier warning of potential flooding within the mud-gas containment vessel.

In still yet another exemplary embodiment, the system includes a discharge valve via which mud is adapted to flow out of one of the first and second vessels; wherein the control unit controls the discharge valve based on at least one of the first and second operating parameters.

In certain exemplary embodiments, each of the first and second sensors is one of the following: a level sensor adapted to measure a fluid level within the first or second vessel; and a pressure sensor adapted to measure pressure within the first or second vessel.

In an exemplary embodiment, the system includes a gas vent line via which the second vessel is in fluid communication with the first vessel; and a third sensor housing assembly connected to the gas vent line, the third sensor housing assembly including a third sensor adapted to measure a third physical property associated with the second vessel; wherein the control unit is in communication with the third sensor to determine and monitor a third operating parameter of the gas vent line; and wherein the third operating parameter is, or is based on, the third physical property.

In another exemplary embodiment, the third operating parameter is selected from the group consisting of: existence of hydrocarbons within the gas vent line; flammables content within the gas vent line; and gas flow rate within the gas vent line.

In yet another exemplary embodiment, the system further includes a flare stack in fluid communication with the gas vent line, the flare stack including an igniter; wherein the control unit controls the operation of the igniter based on the third operating parameter of the gas vent line.

In a third aspect, there is provided a system adapted to monitor at least a first operating parameter of a gas vent line, the system including a sensor housing assembly adapted to be connected to the gas vent line, the sensor housing assembly including a first sensor adapted to measure a first physical property associated with the gas vent line; wherein the monitored first operating parameter is, or is based on, the first physical property measured by the first sensor.

In an exemplary embodiment, the third operating parameter is selected from the group consisting of: existence of hydrocarbons within the gas vent line; flammables content within the gas vent line; and gas flow rate within the gas vent line.

In another exemplary embodiment, the system includes a control unit adapted to be in communication with the first sensor and adapted to receive from the first sensor first measurement data associated with the first physical property; wherein the control unit is adapted to determine the first operating parameter based on the first measurement data.

In yet another exemplary embodiment, the control unit is adapted to be in communication with an electronic drilling recorder (EDR); and wherein the control unit is adapted to send to the EDR first parameter data associated with first operating parameter.

In still yet another exemplary embodiment, the control unit is adapted to control the operation of an igniter of a flare stack, the flare stack being in fluid communication with the gas vent line; wherein the control unit controls the operation of the igniter based on the first operating parameter of the gas vent line.

In certain exemplary embodiments, the sensor housing assembly further includes a first fitting adapted to be connected to the gas vent line, the first fitting defining a first internal passage adapted to be in fluid communication with the gas vent line; a second fitting adapted to be connected to the first vessel, the second fitting defining a second internal passage adapted to be in fluid communication with the gas vent line; and a housing extending between the first and second fittings, the housing defining a second internal region adapted to be in fluid communication with the gas vent line; wherein the first sensor is connected to at least one of the first fitting, the second fitting, and the housing.

In an exemplary embodiment, the sensor housing assembly further includes a second sensor connected to at least one of the first fitting, the second fitting, and the housing; and wherein the second sensor is adapted to measure a second physical property associated with the gas vent line.

In a fourth aspect, there is provided a method according to one or more aspects of the present disclosure.

In a fifth aspect, there is provided a kit according to one or more aspects of the present disclosure.

In a sixth aspect, there is provided an apparatus according to one or more aspects of the present disclosure.

In a seventh aspect, there is provided a sensor housing assembly according to one or more aspects of the present disclosure.

Other aspects, features, and advantages will become apparent from the following detailed description when taken in conjunction with the accompanying drawings, which are a part of this disclosure and which illustrate, by way of example, principles of the inventions disclosed.

### DESCRIPTION OF FIGURES

The accompanying drawings facilitate an understanding of the various embodiments.

FIG. 1 is a diagrammatic illustration of an intelligent sensor system according to an exemplary embodiment, the intelligent sensor system including a sensor housing assembly.

FIG. 2 is a perspective view of a section of the sensor housing assembly of the intelligent sensor system of FIG. 1, according to an exemplary embodiment.

FIG. 3 is a diagrammatic view of the intelligent sensor system of FIG. 1 during operation, according to an exemplary embodiment.

FIG. 4 is a view similar to that of FIG. 3, but depicting the intelligent sensor system of FIG. 1 in communication with an electronic drilling recorder (EDR), according to an exemplary embodiment.

FIG. 5 is a perspective view of a portion of the sensor housing assembly of the intelligent sensor system of FIG. 1, according to another exemplary embodiment.

FIG. 6 is a perspective view of the sensor housing assembly of FIG. 5.

FIG. 7A is a perspective view of a portion of the sensor housing assembly of the intelligent sensor system of FIG. 1, according to yet another exemplary embodiment.

FIG. 7B is a perspective view of the sensor housing assembly of FIG. 7B.

FIG. 7C is a flow chart illustration of a method according to an exemplary embodiment, the method being executed using the intelligent sensor system of FIG. 1, the intelligent sensor system of FIG. 1 including the sensor housing assembly of FIGS. 7A and 7B.

FIG. 8 is a diagrammatic illustration of the intelligent sensor system of FIG. 1 during operation and according to an exemplary embodiment, the intelligent sensor system of FIG. 1 including either the sensor housing assembly of FIGS. 5 and 6 or the sensor housing assembly of FIGS. 7A and 7B.

FIG. 9A is a perspective view of a sensor housing assembly of the intelligent sensor system of FIG. 1, according to still yet another exemplary embodiment.

FIG. 9B is a flow chart illustration of a method according to an exemplary embodiment, the method being executed using the intelligent sensor system of FIG. 1, the intelligent sensor system of FIG. 1 including the sensor housing assembly of FIG. 9A.

FIG. 10 is a diagrammatic view of a portion of an intelligent sensor system, according to an exemplary embodiment.

FIG. 11 is a diagrammatic illustration of a system located at a drilling rig site, according to an exemplary embodiment.

FIG. 12 is a diagrammatic illustration of a portion of the system of FIG. 11, according to an exemplary embodiment.

FIGS. 13A and 13B are elevational views of a sensor housing assembly of the intelligent sensor system of FIG. 1, according to still yet another exemplary embodiment.

FIG. 13C is a sectional view taken along line 13C-13C of FIG. 13B, according to an exemplary embodiment.

FIG. 14A is an elevational view of a sensor housing assembly of the intelligent sensor system of FIG. 1, according to still yet another exemplary embodiment.

FIG. 14B is a sectional view taken along line 14B-14B of FIG. 14A, according to an exemplary embodiment.

FIG. 15 is a diagrammatic illustration of a computing device for implementing one or more exemplary embodiments of the present disclosure, according to an exemplary embodiment.

### DETAILED DESCRIPTION

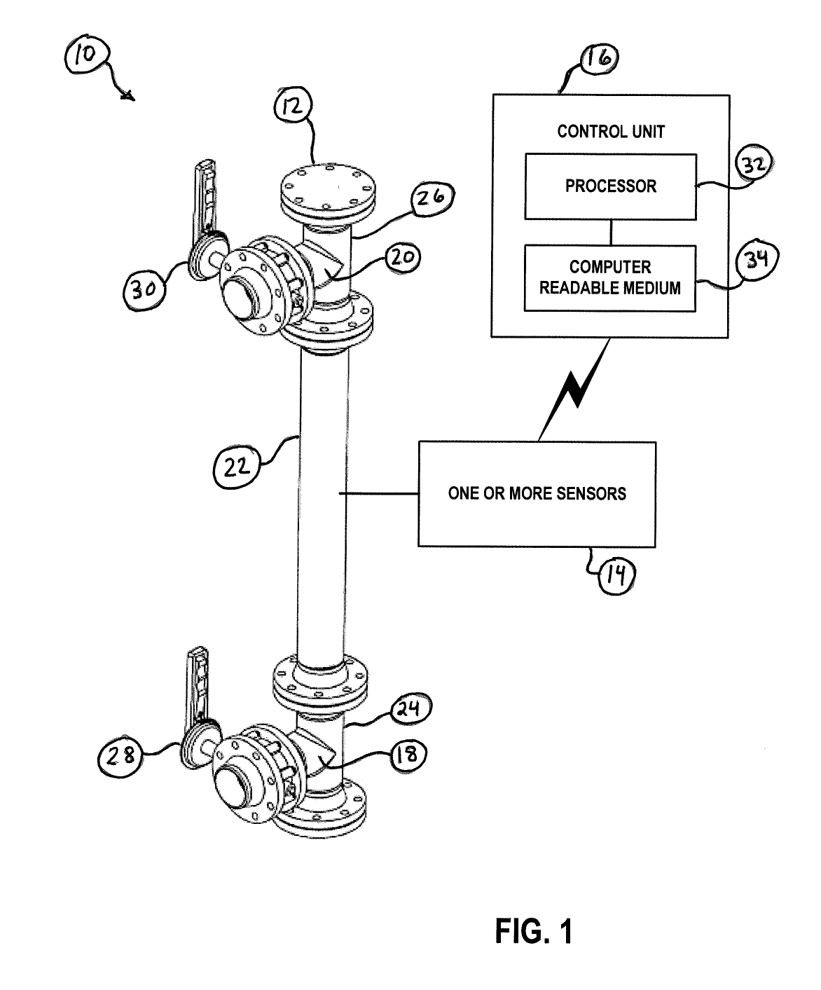

In an exemplary embodiment, as illustrated in FIG. 1, an intelligent sensor system is generally referred to by the reference numeral 10 and includes a sensor housing assembly 12, which includes one or more sensors 14. A control unit 16 is in communication with the one or more sensors 14. The sensor housing assembly 12 includes fittings 18 and 20, and a tubular housing 22 extending therebetween. The fittings 18 and 20 are part of t-fittings 24 and 26, respectively. The tubular housing 22 is connected to, and extends between, the t-fittings 24 and 26. Isolation valves 28 and 30 are connected to the fittings 18 and 20, respectively. The one or more sensors 14 are adapted to measure one or more physical properties associated with a vessel such as, for example, an overflow tank, a mud-gas separator vessel, or a shale-gas separator vessel; the sensor housing assembly 12 is adapted to be connected to this vessel. The control unit 16 includes a processor 32 and a non-transitory computer readable medium 34 operably coupled thereto; a plurality of instructions are stored on the non-transitory computer readable medium 34, the instructions being accessible to, and executable by, the processor 32.

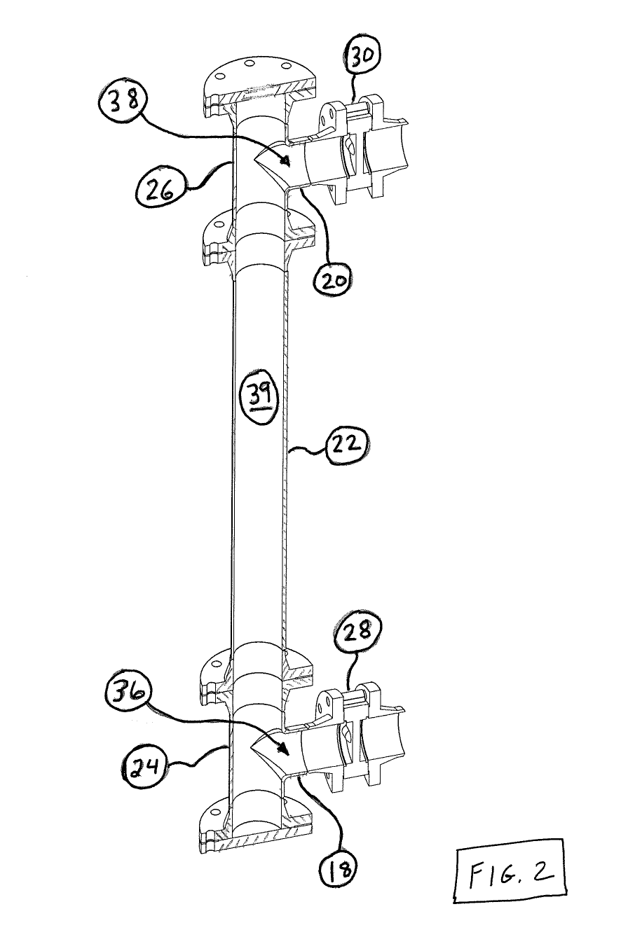

In an exemplary embodiment, as illustrated in FIG. 2 with continuing reference to FIG. 1, the fittings 18 and 20 define internal passages 36 and 38, respectively. The tubular housing 22 defines an internal region 39.

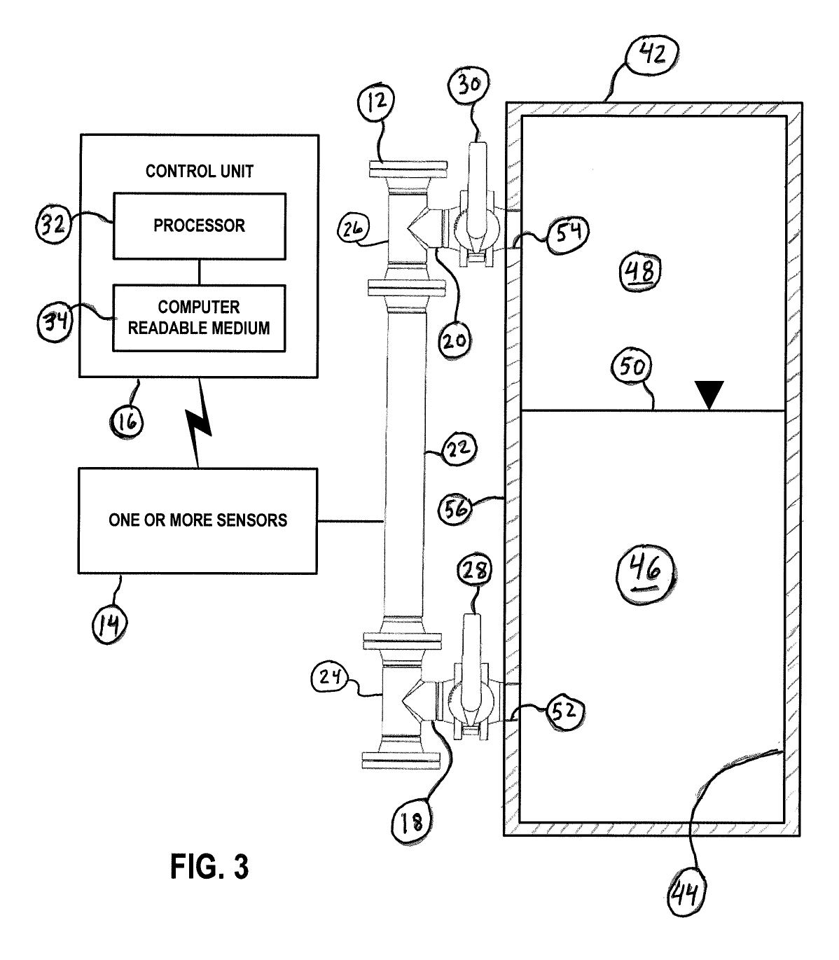

In an exemplary embodiment, as illustrated in FIG. 3 with continuing reference to FIGS. 1 and 2, the sensor housing assembly 12 is connected to a vessel 42. An internal region 44 is defined by the vessel 42. One or more fluids are disposed within the internal region 44; in an exemplary embodiment, these one or more fluids include liquid materials 46 and gas materials 48. In several exemplary embodiments, the vessel 42 may also contain solid materials, which together with the liquid materials 46 form a slurry, or mud, disposed within internal region 44. A fluid level 50 is defined by at least the liquid materials 46; in several exemplary embodiments, the fluid level 50 varies. In several exemplary embodiments, the vessel 42 is adapted to receive a multiphase flow and thus materials having different phases (solid, liquid, and gas) are disposed within the internal region 44.

As shown in FIG. 3, when the sensor housing assembly 12 is connected to the vessel 32, the fittings 18 and 20 are connected to the vessel 42 via the valves 28 and 30, respectively. The internal passages 36 and 38 of the fittings 18 and 20, respectively, are in fluid communication with the internal region 44 via the valves 28 and 30, respectively, and via ports 52 and 54, respectively, which ports are formed in a side wall 56 of the vessel 42. The internal region 39 of the tubular housing 22 is in fluid communication with the internal region 44 of the vessel 42 via the internal passages 26 and 28, as well as other internal passages of the t-fittings 24 and 26, the valves 28 and 30, and the ports 52 and 54. The port 54 is located vertically higher than the port 52. The tubular housing 22 extends vertically, in a generally parallel orientation to the side wall 56 of the vessel 42. In an exemplary embodiment, the sensor housing assembly 12 extends along a portion of the height of the vessel 42. In an exemplary embodiment, the sensor housing assembly 12 extends along the entire, or almost the entire, height of the vessel 42.

In several exemplary embodiments, the vessel 42 is, for example: a mud-gas containment vessel described in U.S. application Ser. No. 13/000,964, filed Jun. 30, 2008, now U.S. Pat. No. 8,641,811, issued Feb. 4, 2014; a catch tank described in U.S. application Ser. No. 13/000,964, filed Jun. 30, 2008, now U.S. Pat. No. 8,641,811, issued Feb. 4, 2014; a mud-gas separator vessel described in U.S. Application No. 62/089,913, filed Dec. 10, 2014; or a shale-gas separator vessel described in U.S. application Ser. No. 14/049,726, filed Oct. 9, 2013, now U.S. Pat. No. 8,784,545, issued Jul. 22, 2014.

In operation, in an exemplary embodiment, via the ports 52 and 54, and the fittings 18 and 20, a portion of at least the liquid materials 46 is disposed within the t-fitting 24, within the t-fitting 24 and the internal region 39, or within the t-fitting 24, the internal region 39, and the t-fitting 26. In some cases, a portion of at least the gas materials 48 is disposed within one or more of the internal region 39 and the t-fittings 24 and 26. Other portions of other materials contained within the vessel 42 may also be disposed within one or more of the internal region 39 and the t-fittings 24 and 26. The one or more sensors 14 measure one or more physical properties associated with the vessel 42. The system 10 then determines one or more operating parameters of the vessel 42; the one or more operating parameters are, or are based on, the one or more physical properties measured by the one or more sensors 14. In an exemplary embodiment, the control unit 16 receives from the one or more sensors 14 measurement data associated with the one or more physical properties measured by the one or more sensors 14. The control unit 16 then processes the measurement data to determine the one or more operating parameters of the vessel 42. In an exemplary embodiment, the control unit 16 is part of the one or more sensors 14.

In several exemplary embodiments, the system 10 provides an intelligent sensor system in which operating parameters of the vessel 42 are determined for the purpose of monitoring the operating parameters.

In several exemplary embodiments, the system 10 provides an early warning of an upset condition that may negatively impact the operation of the vessel 42; such a negative impact may include, for example, a rapid increase in the fluid level 50 and the flooding of the vessel 42.

In several exemplary embodiments, the sensor housing assembly 12 includes one or more alarms, which are in communication with the one or more sensors 14 and/or the control unit 16; the one or more alarms may be audio and/or visual alarms. In an exemplary embodiment, the control unit 16 determines that the determined one or more operating parameters are outside of a predetermined range (or ranges) of values, or are otherwise unacceptable, and triggers the one or more alarms to alert operators. In an exemplary embodiment, the one or more sensors 14 determine that the determined one or more operating parameters are outside of a predetermined range (or ranges) of values, or are otherwise unacceptable, and trigger the one or more alarms to alert operators.

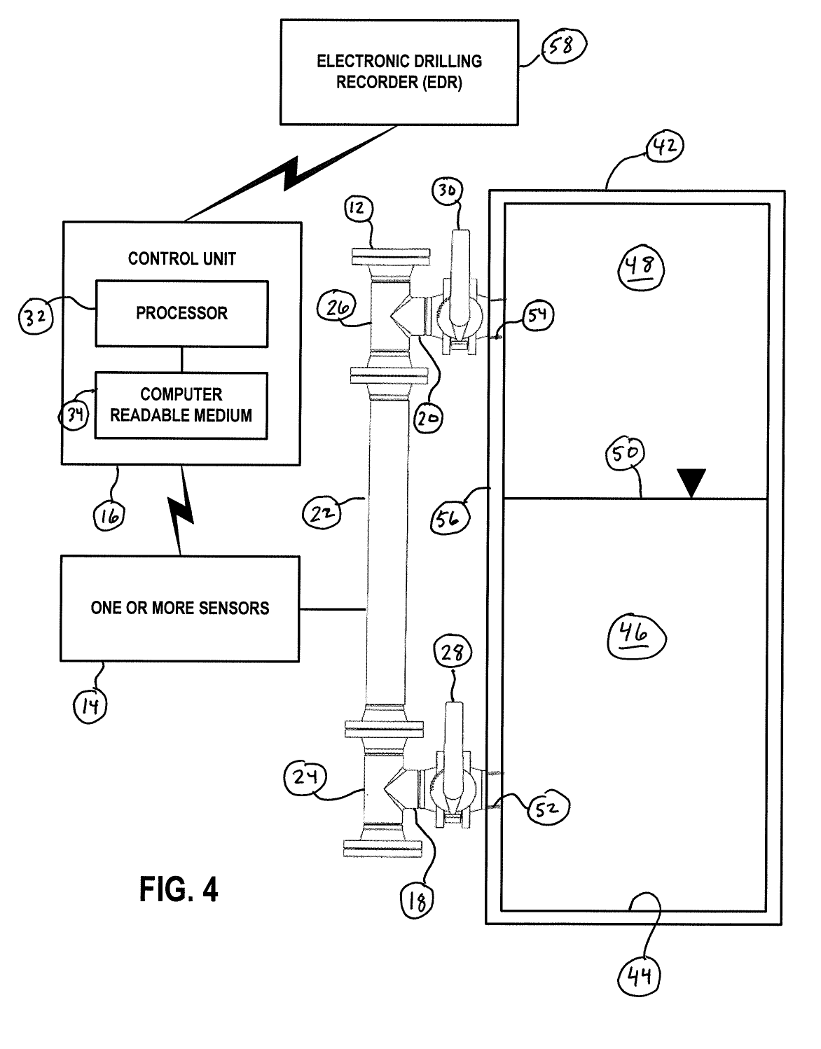

In an exemplary embodiment, as illustrated in FIG. 4 with continuing reference to FIGS. 1-3, an electronic drilling recorder (EDR) 58 is in communication with the control unit 16. The EDR 58 is located at a drilling rig site used in oil and gas exploration and production operations. During the above-described operation of the system 10, the control unit 16 sends to the EDR 58 parameter data associated with the determined one or more operating parameters. Thus, the one or more operating parameters of the vessel 42 are remotely monitored, using the EDR 58, from a central location at the rig site. In an exemplary embodiment, the parameter data sent by the control unit 16 to the EDR 58 includes parameter data indicative of an alarm to trigger operators of the EDR 58, notifying the operators of an upset condition with respect to the vessel 42. In an exemplary embodiment, the control unit 16 is in communication with the EDR 58 via Wellsite Information Transfer Specification (WITS) protocol, enabling remote monitoring and alarm settings.

In an exemplary embodiment, with continuing reference to FIGS. 1-4, instead of, or in addition to, the EDR 58, the control unit 16 is in communication with one or more other computing devices. These one or more other computer devices may be located at either the rig site or another location that is more remote from the vessel 42.

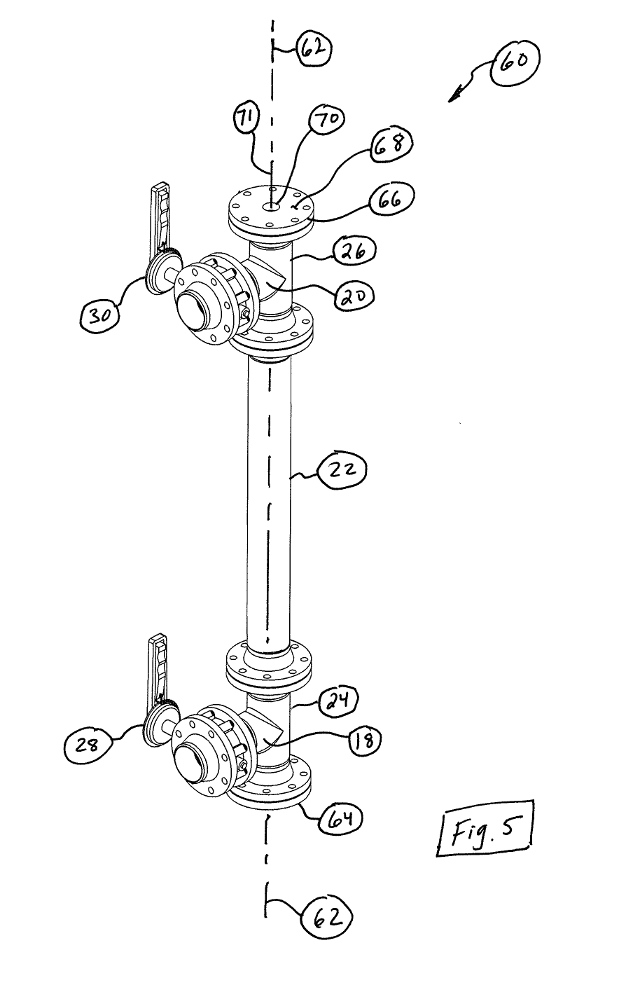

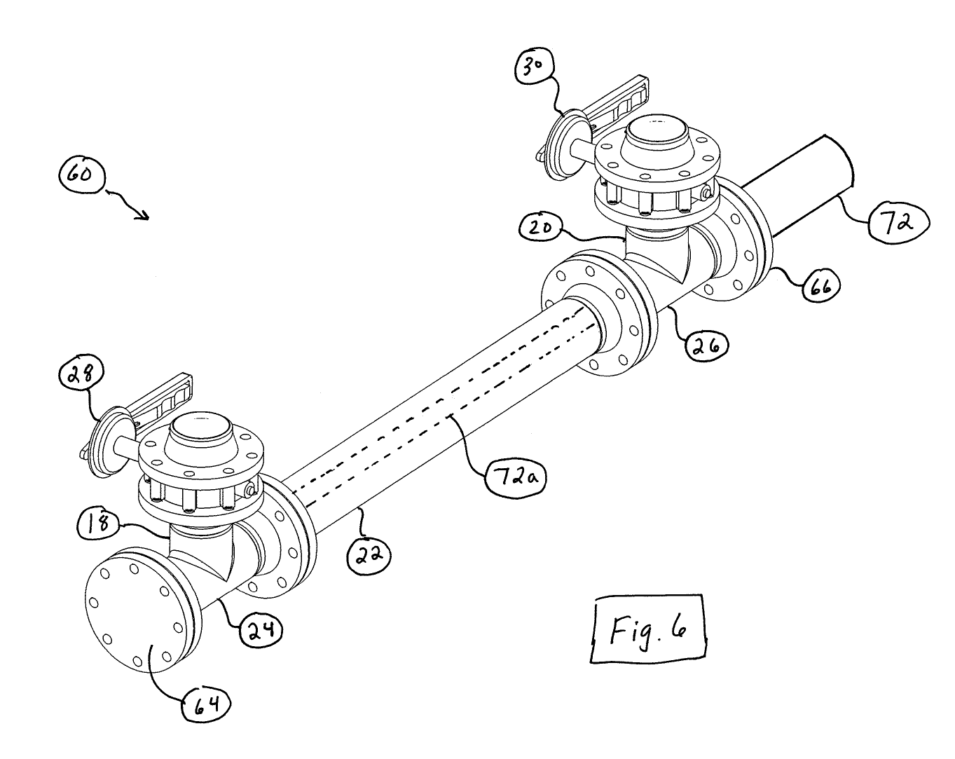

In an exemplary embodiment, as illustrated in FIGS. 5 and 6, the system 10 includes another exemplary embodiment of the sensor housing assembly 12 of FIG. 1, which is generally referred to by the reference numeral 60. The sensor housing assembly 60 of FIGS. 5 and 6 includes all of the components of the sensor housing assembly 12 of FIG. 1, which components are given the same reference numerals. In the sensor housing assembly 60 of FIGS. 5 and 6, the tubular housing defines a longitudinally-extending center axis 62. A solid cap 64 is connected to the t-fitting 24 at the bottom thereof. A cap 66 is connected to the t-fitting 26. The cap 66 lies in a plane 68, which is perpendicular to the longitudinally-extending center axis 62. A port 70 is formed through the cap 66, and is in fluid communication with the internal region 39 of the tubular housing 22. In an exemplary embodiment, the port 70 defines a center axis 71, which is coaxial with the center axis 62.

As shown in FIG. 6, a level sensor 72 is connected to the cap 66 and is positioned, relative to the port 70, so that the level sensor 72 can measure the fluid level 50 within the vessel 42 when the sensor housing assembly 60 is connected thereto. The level sensor 72 is, or is part of, the one or more sensors 14. In an exemplary embodiment, the level sensor 72 is a guided wave level sensor and includes a rod-shaped probe 72a, which extends through the port 70 and within the internal region 39 of the tubular housing 22, and is adapted to contact the liquid materials 46. In an exemplary embodiment, the level sensor 72 is a non-contact radar level sensor and thus the level sensor 72 does not include the rod-shaped probe 72a; instead, at least a portion of the level sensor 72 is positioned adjacent the port 70 and, in some exemplary embodiments, a portion of the level sensor 72 extends through the port 70 but is not adapted to contact the liquid materials 46. The level sensor 72 is in communication with the control unit 16 shown in FIGS. 1, 3, and 4.

In operation, with continuing reference to FIGS. 1-6, in an exemplary embodiment, the sensor housing assembly 60 of FIGS. 5 and 6 is connected to the vessel 42 in the same manner in which the sensor housing assembly 12 of FIG. 1 is connected to the vessel 42. The level sensor 72 measures the fluid level 50 within the internal region 44 of the vessel 42. In an exemplary embodiment, the control unit 16 receives from the level sensor 72 fluid level measurement data associated with the fluid level 50. The control unit 16 then processes the fluid level measurement data to determine one or more operating parameters of the vessel 42. The determined one or more operating parameters of the vessel 42 may include: the actual value of the fluid level 50 itself, the fluid level 50 being at a high level, the fluid level 50 being at a low level, the fluid level 50 undergoing a rapid level change (increasing or decreasing), or any combination thereof. In several exemplary embodiments, the control unit 16 and/or the level sensor 72 provide high level, low level, and rapid-level change alarms (audible and/or visible) to alert operators. In several exemplary embodiments, the control unit 16 is in communication with the EDR 58 and/or one or more other remotely-located computing devices, sending to these devices fluid level parameter data associated with the determined one or more operating parameters of the vessel 42, thereby enabling remote monitoring of the one or more operating parameters of the vessel 42.

In several exemplary embodiments, the perpendicular orientation between the center axis 62 and the plane 68 in which the cap 66 lies facilitates the measurement of the fluid level 50 by the level sensor 72 when the level sensor 72 is a guided wave level sensor and thus includes the probe 72a; in such an embodiment, the probe 72a easily extends through the port 70 and into the internal region 39, facilitating the measurement of the fluid level 50. In several exemplary embodiments, the perpendicular orientation between the center axis and the plane 68 in which the cap 66 lies facilitates the measurement of the fluid level 50 by the level sensor 72 when the level sensor 72 is a non-contact radar level sensor; in such an embodiment, the non-contact radar level sensor transmits radar waves in a direction that is perpendicular to the fluid level 50 within the internal region 39, facilitating the measurement of the fluid level 50.

In several exemplary embodiments, the system 10, including the sensor housing assembly 60, provides an intelligent sensor system in which operating parameters associated with the fluid level 50 of the vessel 42 are determined and monitored, on-site or remotely. In several exemplary embodiments, the system 10, including the sensor housing assembly 60, can provide fluid level measurements inside the vessel 42, which can be, for example, a separator vessel or a containment vessel; the measurement of fluid levels enables setting high level, low level, and rapid level change alarms. The alarms may be visual and/or audible and can be in communication with the EDR 58 for remote monitoring. In several exemplary embodiments, the system 10, including the sensor housing assembly 60, can estimate the time until the overflow of the vessel 42.

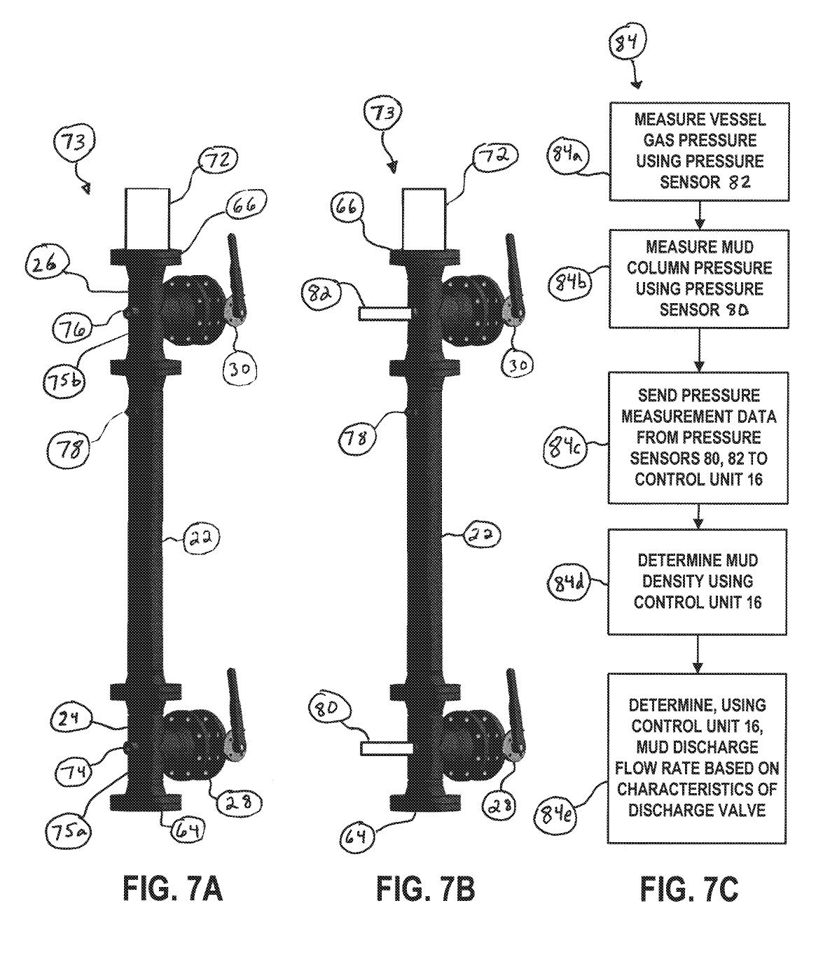

In an exemplary embodiment, as illustrated in FIGS. 7A and 7B with continuing reference to FIGS. 1-6, yet another exemplary embodiment of the sensor housing 12 of FIG. 1 is generally referred to by the reference numeral 73. The sensor housing assembly 73 includes all of the components of the sensor housing 60 of FIGS. 5 and 6, which identical components are given the same reference numerals. In addition to the components of the sensor housing assembly 60, the sensor housing assembly 73 of FIGS. 7A and 7B further includes a port 74 formed at a lower end portion 75a of the sensor housing assembly 73, a port 76 formed at an opposing upper end portion 75b of the sensor housing assembly 73, and a port 78 formed between the lower and upper end portions 75a, 75b of the sensor housing assembly 73. Each of the ports 74, 76, and 78 is in fluid communication with the internal region 39 of the tubular housing 22. As shown in FIG. 7A, the ports 74 and 76 are formed in the t-fittings 24 and 26, respectively; in several exemplary embodiments, the ports 74 and 76 are instead formed in the tubular housing 22.

As shown in FIG. 7B, a pressure sensor 80 is positioned adjacent the port 74 and is adapted to measure, via the port 74, mud column pressure within the vessel 42, that is, the pressure of the column of the slurry, or mud, disposed within the internal region 44 of the vessel 42 (the slurry or mud includes the liquid materials 46). A pressure sensor 82 is positioned adjacent the port 76 and is adapted to measure, via the port 76, the vessel gas pressure within the vessel 42, that is, the pressure of the gas materials 48 within the internal region 44 of the vessel 42. The pressure sensors 80 and 82 are part of the one or more sensors 14. The port 78 is a water jet port that is adapted to enable cleaning of the tubular housing 22 and the rod 72a of the level sensor 72 if there is mud deposition and/or plugging within the tubular housing 22. During the operation of the sensor housing assembly 73, the port 78 is normally plugged or otherwise sealed off from the surrounding environment.

In several exemplary embodiments, the operation of the sensor housing assembly 73 is identical to that of the sensor housing assembly 60 except that, in addition to measuring the fluid level 50 using the level sensor 72, the sensor housing assembly 73 also measures respective pressures using the pressure sensors 80 and 82. As a result, the operating parameters of the vessel 42, which are determined by the system 10, may be based on the measurement of the fluid level 50 taken by the level sensor 72, the pressure measurement taken by the pressure sensor 80, the pressure measurement taken by the pressure sensor 82, or any combination thereof.

In an exemplary embodiment, as illustrated in FIG. 7C with continuing reference to FIGS. 1-7B, a method is generally referred to by the reference numeral 84. The method 84 is executed during the operation of the sensor housing assembly 73. The method 84 includes step 84a, at which the vessel gas pressure within the vessel 42 is measured using the pressure sensor 82. Before, during, or after the step 84a, at step 84b the mud column pressure within the vessel 42 is measured using the pressure sensor 80. Before, during, or after the step 84b, at step 84c pressure measurement data associated with the mud column pressure and the vessel gas pressure are sent from the pressure sensors 80 and 82 to the control unit 16. During or after the step 84c, at step 84d the mud density is determined using the control unit 16, the determination of the mud density being based on the pressure measurement data sent from the pressure sensors 80 and 82.

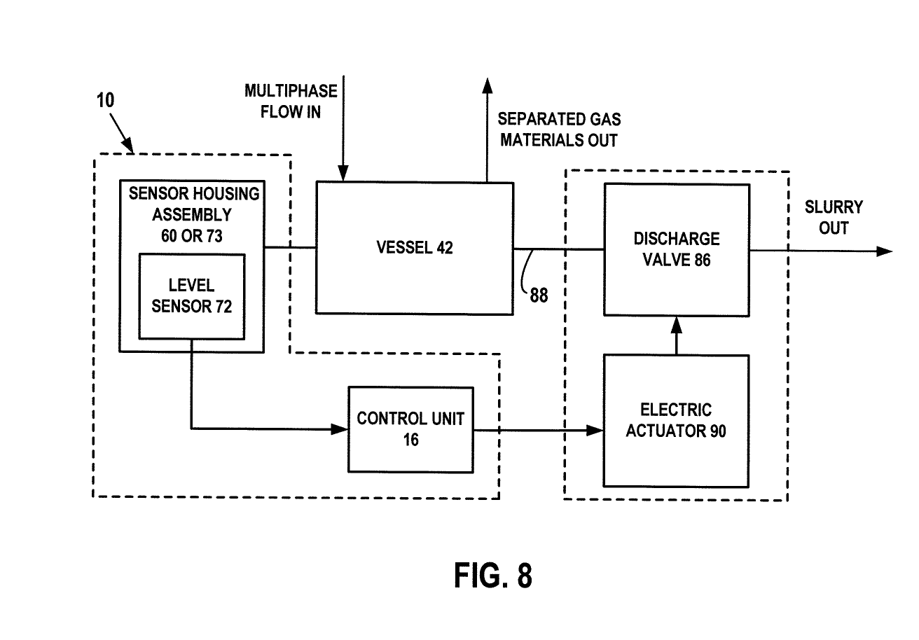

In several exemplary embodiments, the vessel 42 includes, or is connected to, a discharge valve 86 (shown in FIG. 8), via which the slurry or mud is being discharged from the vessel 42; if the vessel 42 includes the discharge valve 86, the method 84 includes step 84e. More particularly, before, during, or after the step 84d, at the step 84e a mud discharge flow rate is determined using the control unit 16, the mud discharge flow rate being based on the pressure measurement data sent from the pressure sensors 80 and 82, as well as the characteristics of the discharge valve 86 via which the slurry or mud is being discharged from the internal region 44. In several exemplary embodiments, if the vessel 42 does not include the discharge valve 86, the step 84e is omitted and the discharge flow rate is not calculated.

In several exemplary embodiments, instead of, or in addition to, one or more of the mud column pressure, the vessel gas pressure, the mud density, and the mud discharge flow rate, one or more other operating parameters of the vessel 42 are determined using the system 10 with the sensor housing assembly 73.

In several exemplary embodiments, the control unit 16 is in communication with the EDR 58 and/or one or more other remotely-located computing devices, sending to these devices fluid level parameter data and/or pressure level parameter data associated with the determined one or more operating parameters of the vessel 42, thereby enabling remote monitoring of the one or more operating parameters of the vessel 42.

In an exemplary embodiment, as illustrated in FIG. 8 with continuing reference to FIGS. 1-7, the vessel 42 includes, or is connected to, the discharge valve 86. In an exemplary embodiment, a multiphase flow enters the vessel 42, the gas materials 48 flow out of the vessel 42 via a flow path, and remaining solid and liquid materials, the slurry or mud, flow out of the vessel 42 via a flow path 88, which is different from the flow path via which the gas materials 48 flow. The discharge valve 86 is in fluid communication with the flow path 88.

As shown in FIG. 8, the system 10 includes either the sensor housing assembly 60 or the sensor housing assembly 73. The control unit 16 is in communication with the sensor housing 60 or 73, and is also in communication with an electric actuator 90, which is operably coupled to the discharge valve 86. In an exemplary embodiment, the electric actuator 90 is part of the system 10. In an exemplary embodiment, the electric actuator 90 and the discharge valve 86 are part of the system 10. In an exemplary embodiment, the electric actuator 90 is part of the discharge valve 86, and the discharge valve 86 is in communication with the control unit 16 via the electric actuator 90. In an exemplary embodiment, the electric actuator 90 is part of the discharge valve 86, the discharge valve 86 is in communication with the control unit 16 via the electric actuator 90, and the electric actuator 90 and the discharge valve 86 are part of the system 10.

In operation, in several exemplary embodiments, the discharge valve 86 is automatically controlled by the respective operations of the level sensor 72, the control unit 16, and the electric actuator 90.

More particularly, in several exemplary embodiments, over time the fluid level 50 rises, and the level sensor 72 measures the fluid level 50 over this time. When the fluid level 50 reaches a predetermined level, the discharge valve 86 is either opened or opened further, and at least a portion of the slurry is discharged from the vessel 42, flowing out of the vessel 42 via the flow path 88. The slurry subsequently flows through the control valve 74 and additional flow line(s) downstream thereof. The level sensor 72 continues to measures the fluid level 50 and communicates data associated with the measurement to the control unit 16. The control unit 16 reads the data and, in turn, automatically controls the electric actuator 90, which opens, further opens, or further closes the discharge valve 74 based on the measurement data received from the level sensor 72; thus, the control unit 16 automatically controls the discharge valve 86. The automatic control of the discharge valve 86 controls the discharge of the slurry out of the vessel 42. In several exemplary embodiments, based on the measurement data received from the level sensor 72, the control unit 16: opens or further opens the discharge valve 86, allowing more slurry to flow out of the internal region 44 and thus reducing the fluid level 50; further closes the discharge valve 86, reducing the amount of slurry that flows out of the internal region 44 and thus increasing the fluid level 50; or maintains the current valve position of the discharge valve 86, the current valve position of the discharge valve 86 being at a fully open valve position, a fully closed valve position, or a partially open valve position. As a result, the fluid level 50 can be automatically maintained within a predetermined range, or at a predetermined value, within the vessel 42. As result, vent gas carry under is prevented. Also as a result, the slurry, or at least the liquid materials 46, are prevented from filling up the vessel 42, overflowing and flooding the vessel 42.

In several exemplary embodiments, during the above-described operation of the system 10 and the vessel 42, including the operation of the electric actuator 90 and the discharge valve 86, the control unit 16 determines the slurry discharge flow rate using the fluid level measurement data sent by the level sensor 72 to the control unit 16. In several exemplary embodiments, the control unit 16 also determines liquid weight using measurement data received from at least the level sensor 72. In several exemplary embodiments, if the control unit 16 is in communication with the sensor housing assembly 73 (rather than with the sensor housing assembly 60), the control unit 16 determines liquid weight and/or one or more other operating parameters of the vessel 42 using measurement data received from one or more of the level sensor 72, the pressure sensor 80, and the pressure sensor 82.

In several exemplary embodiments, the combination of the level sensor 72, the control unit 16, the electric actuator 90, and the discharge valve 86 provides intelligent system control of slurry discharge from the vessel 42, thereby actively controlling the fluid level 50 and actively preventing vent gas carry under, as well as slurry or liquid overflow.

In several exemplary embodiments, the control unit 16 may include one or more alarms, and during operation may activate the one or more alarms when the fluid level 50 is too high (i.e., is at, or exceeds, a predetermined high level). In several exemplary embodiments, during operation, the control unit 16 may activate one or more alarms when the fluid level 50 is too low (i.e., is at, or is below, another predetermined low level). Instead of, or in addition to, activating one or more alarms, the control unit 16 may take other action(s) when the fluid level 50 is too high or too low.

In several exemplary embodiments, the control unit 16 is in communication with the EDR 58 and/or one or more other remotely-located computing devices, sending to these devices fluid level parameter data and/or pressure level parameter data associated with the determined one or more operating parameters of the vessel 42, thereby enabling remote monitoring of the one or more operating parameters of the vessel 42.

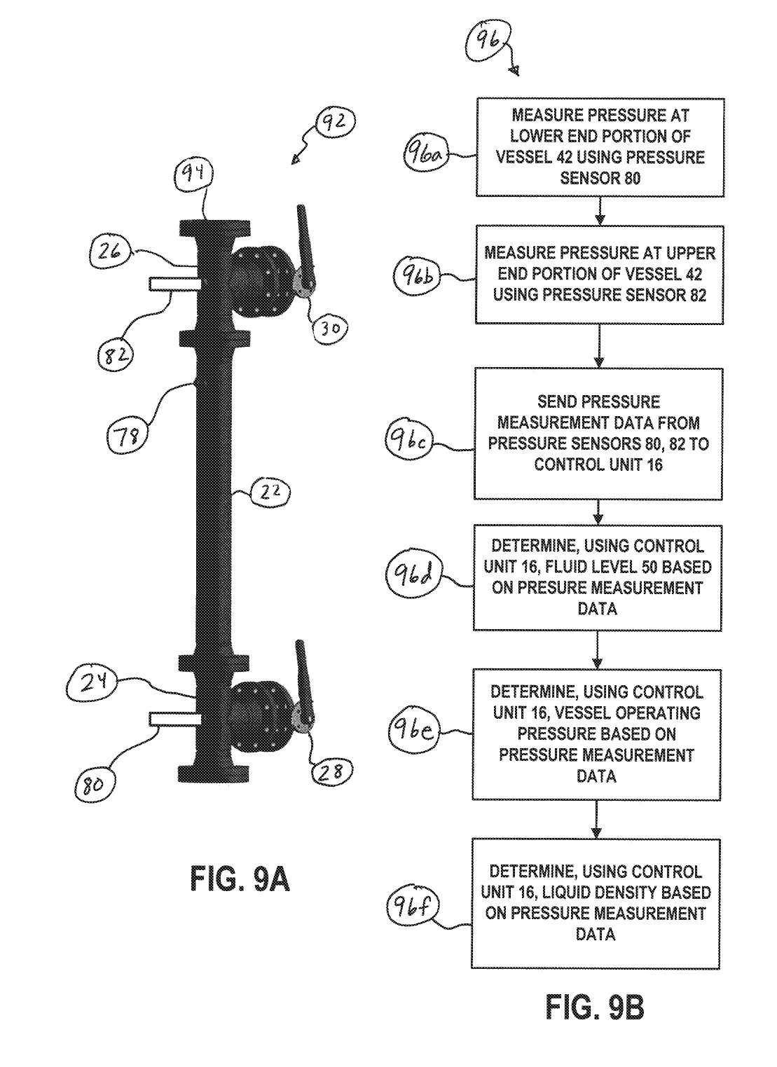

In an exemplary embodiment, as illustrated in FIG. 9A with continuing reference to FIGS. 1-8, still yet another exemplary embodiment of the sensor housing assembly 12 of FIG. 1 is generally referred to by the reference numeral 92. The sensor housing assembly 92 is identical to the sensor housing assembly 73 of FIGS. 7A and 7B except that the level sensor 72, the cap 66, and the port 70 are omitted from the sensor housing assembly 92. In further contrast to the sensor housing assembly 73, and instead of the level sensor 72, the cap 66, and the port 70, the sensor housing assembly 92 includes a solid cap 94, which is connected to the t-fitting 26 at the top thereof. The sensor housing assembly 92 is part of the system 10, with the pressure sensors 80 and 82 in communication with the control unit 16.

In operation, with continuing reference to FIGS. 1-9A, in an exemplary embodiment, the sensor housing assembly 92 of FIG. 9A is connected to the vessel 42 in the same manner in which the sensor housing assembly 12 of FIG. 1 is connected to the vessel 42. The pressure sensor 80 measures pressure at the lower end portion of the vessel 42. The pressure sensor 82 measures pressure at the upper end portion of the vessel 42. The control unit 16 receives from the pressure sensors 80 and 82 pressure measurement data associated with the respective pressures at the lower end portion and the upper end portion of the vessel 42. The control unit 16 then processes the pressure measurement data to determine one or more operating parameters of the vessel 42. The determined one or more operating parameters of the vessel 42 may include: the fluid level 50, the operating pressure of the vessel 42, the liquid density, or any combination thereof. In several exemplary embodiments, the control unit 16 provides high pressure alarms (audible and/or visible) to alert operators. The alarms can be in communication with the EDR 58 for remote monitoring.

In several exemplary embodiments, the control unit 16 is in communication with the EDR 58 and/or one or more other remotely-located computing devices, sending to these devices pressure parameter data associated with the determined one or more operating parameters of the vessel 42, thereby enabling remote monitoring of the one or more operating parameters of the vessel 42.

In several exemplary embodiments, the system 10, including the sensor housing assembly 92, provides an intelligent sensor system in which operating parameters associated with pressure within the vessel 42 are determined and monitored, on-site or remotely.

In an exemplary embodiment, as illustrated in FIG. 9B with continuing reference to FIGS. 1-9A, a method is generally referred to by the reference numeral 96. The method 96 is executed during the above-described operation of the sensor housing assembly 92. The method 96 includes step 96a, at which the pressure at the lower end of the vessel 42 is measured using the pressure sensor 80. Before, during, or after the step 96a, the pressure at the upper end portion of the vessel 42 is measured using the pressure sensor 82. Before, during, or after the step 96b, at step 96c pressure measurement data associated with the respective pressures at the lower and upper end portions of the vessel 42 are sent from the pressure sensors 80 and 82 to the control unit 16. During or after the step 96c, at step 96d the fluid level 50 is determined using the control unit 16, the determination of the fluid level 50 being based on the pressure measurement data sent from the pressure sensors 80 and/or 82. During or after the step 96d, at step 96e the vessel operating pressure is determined using the control unit 16, the determination of the vessel operating pressure being based on the pressure measurement data sent from the pressure sensors 80 and/or 82. During or after the step 96e, at step 96f the liquid density is determined using the control unit 16, the determination of the liquid density being based on the pressure measurement data sent from the pressure sensors 80 and/or 82.

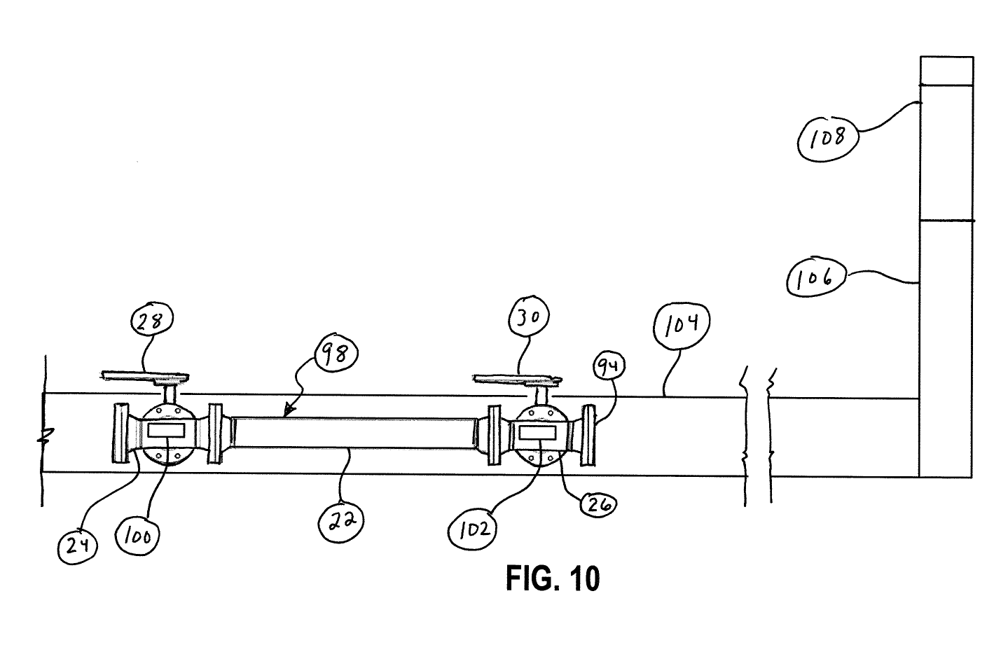

In an exemplary embodiment, as illustrated in FIG. 10 with continuing reference to FIGS. 1-9, still yet another exemplary embodiment of the sensor housing assembly 12 of FIG. 1 is generally referred to by the reference numeral 98. The sensor housing assembly 98 is identical to the sensor housing 92 of FIG. 9A, except that the pressure sensors 80 and 82 are omitted in favor of sensors 100 and 102, respectively. Each of the sensors 100 and 102 are adapted to measure physical properties associated with a gas vent line, such as gas vent line 104 illustrated in FIG. 10. The tubular housing 22 extends horizontally. The sensor housing assembly 98 is part of the system 10, and the sensors 100 and 102 are in communication with the control unit 16. The sensors 100 and 102 are part of the one or more sensors 14.

In operation, with continuing reference to FIGS. 1-10, in an exemplary embodiment, the sensor housing assembly 98 is connected to the gas vent line 104 so that each of the internal region 39, the internal passage 36, and the internal passage 38 is in fluid communication with the gas vent line 104. In an exemplary embodiment, the fittings 18 and 20 are connected to the gas vent line 104 via the valves 28 and 30, respectively. The sensors 100 and 102 measure physical properties associated with the gas vent line 104 such as, for example, the existence of hydrocarbons in the gas vent line 104, the flammables content within the gas vent line 104, the gas flow rate in the gas vent line 104, or any combination thereof. The control unit 16 then processes the measurement data to determine one or more operating parameters of the gas vent line 104. The determined one or more operating parameters of the gas vent line 104 may include: the existence of hydrocarbons in the gas vent line 104, the flammables content within the gas vent line 104, the gas flow rate in the gas vent line 104, or any combination thereof. In several exemplary embodiments, the control unit 16 provides high pressure alarms (audible and/or visible) to alert operators. The alarms can be in communication with the EDR 58 for remote monitoring.

In several exemplary embodiments, the control unit 16 is in communication with the EDR 58 and/or one or more other remotely-located computing devices, sending to these devices vent line parameter data associated with the determined one or more operating parameters of the gas vent line 104, thereby enabling remote monitoring of the one or more operating parameters of the gas vent line 104.

In several exemplary embodiments, the system 10, including the sensor housing assembly 98, provides an intelligent sensor system in which operating parameters associated with the gas vent line 104 are determined and monitored, on-site or remotely.

In an exemplary embodiment, a flare stack 106 is in fluid communication with the gas vent line 104, and includes an igniter 108. In an exemplary embodiment, during operation, the control unit 16 automatically controls the operation of the igniter 108 based on the determined operating parameters of the gas vent line 104. Thus, the system 10 provides for the intelligent automation of the igniter 108.

In several exemplary embodiments, the gas vent line 104 extends vertically and the sensor housing assembly 98 also extends vertically.

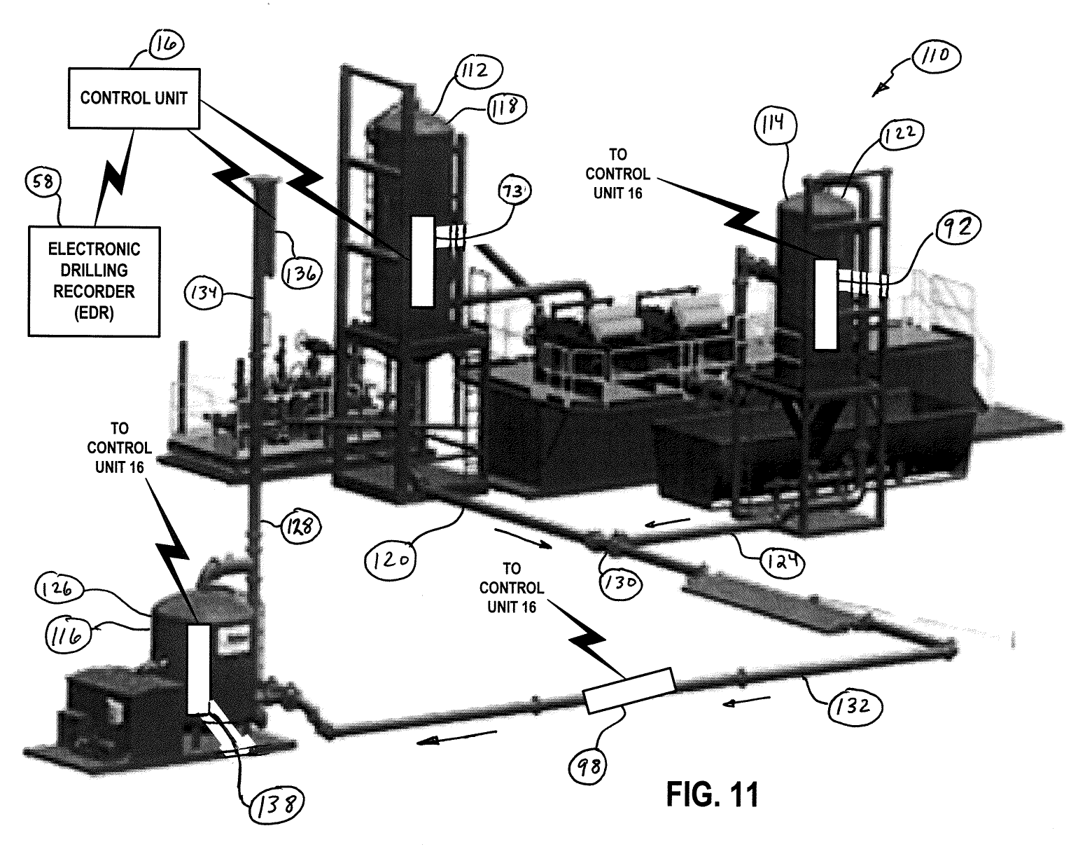

In an exemplary embodiment, as illustrated in FIG. 11 with continuing reference to FIGS. 1-10, a system is generally referred to by the reference numeral 110. The system 110 is located on an oil and gas drilling rig site, and is used during oil and gas exploration and production operations. The system 110 includes the control unit 16, the EDR 58, a mud-gas separator system 112, a shale-gas separator system 114, and a mud-gas containment system 116. The mud-gas separator system 112 includes a mud-gas separator vessel 118, and a gas vent line 120. The shale-gas separator system 114 includes a shale-gas separator vessel 122, and a gas vent line 124. The mud-gas containment system 116 includes a mud-gas containment vessel 126, and a gas vent line 128. The gas vent lines 120 and 124 are connected together at a joint 130. A gas vent line 132 is connected to the joint 130, and extends to the mud-gas containment vessel 126. The mud-gas separator vessel 118 is in fluid communication with the mud-gas containment vessel 126 via at least the gas vent line 120, the joint 130, and the gas vent line 132. The shale-gas separator vessel 122 is in fluid communication with the mud-gas containment vessel 126 via at least the gas vent line 120, the joint 130, and the gas vent line 132.

The mud-gas containment system 116 further includes a flare stack 134, which is connected to, and in fluid communication with, the gas vent line 128. The flare stack 134 includes an igniter 136. The igniter 136 is in communication with the control unit 16. The flare stack 134 is in fluid communication with the gas vent line 132 via at least the mud-gas containment vessel 126 and the gas vent line 128. In several exemplary embodiments, one or more exemplary embodiments of the mud-gas containment system 116 are described in whole or in part in U.S. application Ser. No. 13/000,964, filed Jun. 30, 2008, now U.S. Pat. No. 8,641,811, issued Feb. 4, 2014.

The mud-gas separator system 112 further includes the discharge valve 86 (not shown in FIG. 11 but shown in FIG. 8), which is fluid communication with an internal region defined by the mud-gas separator vessel 118. The discharge valve 86 is in communication with the control unit 16. In several exemplary embodiments, one or more exemplary embodiments of the mud-gas separator system 112 are described in whole or in part in U.S. Application No. 62/089,913, filed Dec. 10, 2014.

The shale-gas separator system 114 includes a discharge line (not shown), which is in fluid communication with an internal region defined by the shale-gas separator vessel 122. In several exemplary embodiments, one or more exemplary embodiments of the shale-gas separator system 114 are described in whole or in part in U.S. application Ser. No. 14/049,726, filed Oct. 9, 2013, now U.S. Pat. No. 8,784,545, issued Jul. 22, 2014.

The system 110 further includes: the sensor housing assembly 73 of FIGS. 7A and 7B, the sensor housing assembly 73 being connected to the mud-gas separator vessel 118; the sensor housing assembly 92 of FIG. 9A, the sensor housing assembly 92 being connected to the shale-gas separator vessel 122; the sensor housing assembly 98 of FIG. 10, the sensor housing assembly 98 being connected to the gas vent line 132; and a sensor housing assembly 138, the sensor housing assembly 138 being connected to the mud-gas containment vessel 126. The sensor housing assembly 138 is identical to the sensor housing assembly 73 of FIGS. 7A and 7B, and components of the sensor housing assembly 138 will be referred to using the same reference numerals as those used to refer to the corresponding identical components of the sensor housing assembly 73 of FIGS. 7A and 7B.

In operation, in an exemplary embodiment, the mud-gas separator vessel 118 receives a multiphase flow, and separates gas materials from solid and liquid materials in the multiphase flow. The separated gas materials flow out of the mud-gas separator vessel 118 via the gas vent line 120. As necessary or desired, the discharge valve 86 is opened, and at least a portion of the remaining solid and liquid materials flow out of the mud-gas separator vessel 118 via the discharge valve 86. Before, during, or after the separation and discharge operations of the mud-gas separator system 112, the sensor housing assembly 73 of FIGS. 7A and 7B measures the fluid level within the mud-gas separator vessel 118 using the level sensor 72, the mud column pressure within the mud-gas separator vessel 118 using the pressure sensor 80, and the vessel gas pressure within the mud-gas separator vessel 118 using the pressure sensor 82. The sensors 72, 80, and 82 send level and pressure measurement data to the control unit 16, which determines one or more operating parameters of the mud-gas separator vessel 118 based on the measurement data. These determined operating parameters may be monitored at the control unit 16. In several exemplary embodiments, the control unit 16 sends to the EDR 58 parameter data associated with the determined one or more operating parameters. Thus, the one or more operating parameters of the mud-gas separator vessel 118 are remotely monitored, using the EDR 58, from a central location at the rig site at which the system 110 is located. In several exemplary embodiments, the control unit 16 controls the discharge valve 86 based on the determined one or more operating parameters of the mud-gas separator vessel 118, causing the discharge valve 86 to be opened, further opened, less open, or closed.

Before, during, or after the above-described operation of the mud-gas separator system 112 and the sensor housing assembly 73 of FIGS. 7A and 7B, the shale-gas separator vessel 122 receives a multiphase flow, the multiphase flow including at least shale materials and gas materials. The shale-gas separator vessel 122 separates the gas materials from at least the shale materials. The separated gas materials flow out of the shale-gas separator vessel 122 via the gas vent line 124. The remaining shale materials, and in several exemplary embodiments other materials, may flow out of the shale-gas separator vessel 122 via the discharge line (not shown). Before, during, or after the separation and discharge operations of the shale-gas separator system 114, the sensor housing assembly 92 of FIG. 9A measures the pressure at the bottom portion of the shale-gas separator vessel 122 using the pressure sensor 80, and the pressure at the upper portion of the shale-gas separator vessel 122 using the pressure sensor 82. The sensors 80 and 82 send pressure measurement data to the control unit 16, which determines one or more operating parameters of the shale-gas separator vessel 122 based on the pressure measurement data. These determined operating parameters may be monitored at the control unit 16. In several exemplary embodiments, the control unit 16 sends to the EDR 58 parameter data associated with the determined one or more operating parameters. Thus, the one or more operating parameters of the shale-gas separator vessel 122 are remotely monitored, using the EDR 58, from a central location at the rig site at which the system 110 is located.

Before, during, or after the above-described operation of the shale-gas separation system 114 and the sensor housing assembly 92 of FIG. 9A, the separated gas materials flowing in the gas vent lines 120 and 124 flow into the joint 130, and then flow through the gas vent line 132 and into the mud-containment vessel 126. During this flow through the gas vent line 132, the sensors 100 and 102 of the sensor housing assembly 98 of FIG. 10 measure physical properties associated with the gas vent line 132, and send measurement data to the control unit 16. The control unit 16 then processes the measurement data to determine one or more operating parameters of the gas vent line 132. The determined one or more operating parameters of the gas vent line 132 may include: the existence of hydrocarbons in the gas vent line 132, the flammables content within the gas vent line 132, the gas flow rate in the gas vent line 132, or any combination thereof. These determined operating parameters may be monitored at the control unit 16. In several exemplary embodiments, the control unit 16 sends to the EDR 58 parameter data associated with the determined one or more operating parameters. Thus, the one or more operating parameters of the gas vent line 132 are remotely monitored, using the EDR 58, from a central location at the rig site at which the system 110 is located. In an exemplary embodiment, during operation, the control unit 16 automatically controls the operation of the igniter 136 based on the determined operating parameters of the gas vent line 132.

Before, during, or after the above-described operation of the gas vent line 132 and the sensor assembly housing 98 of FIG. 10, the separated gas materials flowing through the gas vent line 132 flow into the mud-gas containment vessel 126. Any solid or liquid materials that still remain in the separated gas materials collect within the mud-gas containment vessel 126. In contrast, the gas materials flow upwards, out of the mud-gas containment vessel 126 and into the gas vent line 128. The gas materials flow through the gas vent line 128 and into the flare stack 134. The flare stack 134, which includes the igniter 136, operates to burn off the gas materials flowing into the flare stack 134. Before, during, or after the further separation of the gas materials from any solid and liquid materials within the mud-gas containment vessel 126, the sensor housing assembly 138 measures the fluid level within the mud-gas containment vessel 126 using the level sensor 72, the internal pressure at the lower end portion of the mud-gas containment vessel 126 using the pressure sensor 80, and the internal pressure at the upper end portion of the mud-gas containment vessel 126 using the pressure sensor 82. The sensors 72, 80, and 82 send level and pressure measurement data to the control unit 16, which determines one or more operating parameters of the mud-gas containment vessel 126 based on the measurement data. These determined operating parameters may be monitored at the control unit 16. In several exemplary embodiments, the control unit 16 sends to the EDR 58 parameter data associated with the determined one or more operating parameters. Thus, the one or more operating parameters of the mud-gas containment vessel 126 are remotely monitored, using the EDR 58, from a central location at the rig site at which the system 110 is located.

In several exemplary embodiments, the sensor housing assembly 138, in combination with the control unit 16, enables level measurement of the mud-gas containment vessel 126. In several exemplary embodiments, alarms may be set using the sensor housing assembly 138 and/or the control unit 16 so that the audible and/or visual alarm(s) may be triggered when the fluid level is too high or too low within the mud-gas containment vessel 126. In several exemplary embodiments, a rapid-level-change alarm may be set using the sensor housing assembly 138 and/or the control unit 16, improving response time, that is, increasing the amount of time available to operators to respond to the condition that triggered the alarm. In several exemplary embodiments, the sensor housing assembly 138, in combination with the control unit 16, provides an early warning of any flooding of the mud-gas containment vessel 126. In several exemplary embodiments, the sensor housing assembly 138, in combination with the control unit 16, provides the fill rate within the mud-gas containment vessel 126, the fill rate being part of the determined one or more operating parameters of the mud-gas containment vessel 126. In several exemplary embodiments, the sensor housing assembly 138, in combination with the control unit 16, provides monitoring of vessel pressure and liquid density, the vessel pressure and liquid density being part of the determined one or more operating parameters of the mud-gas containment vessel 126.

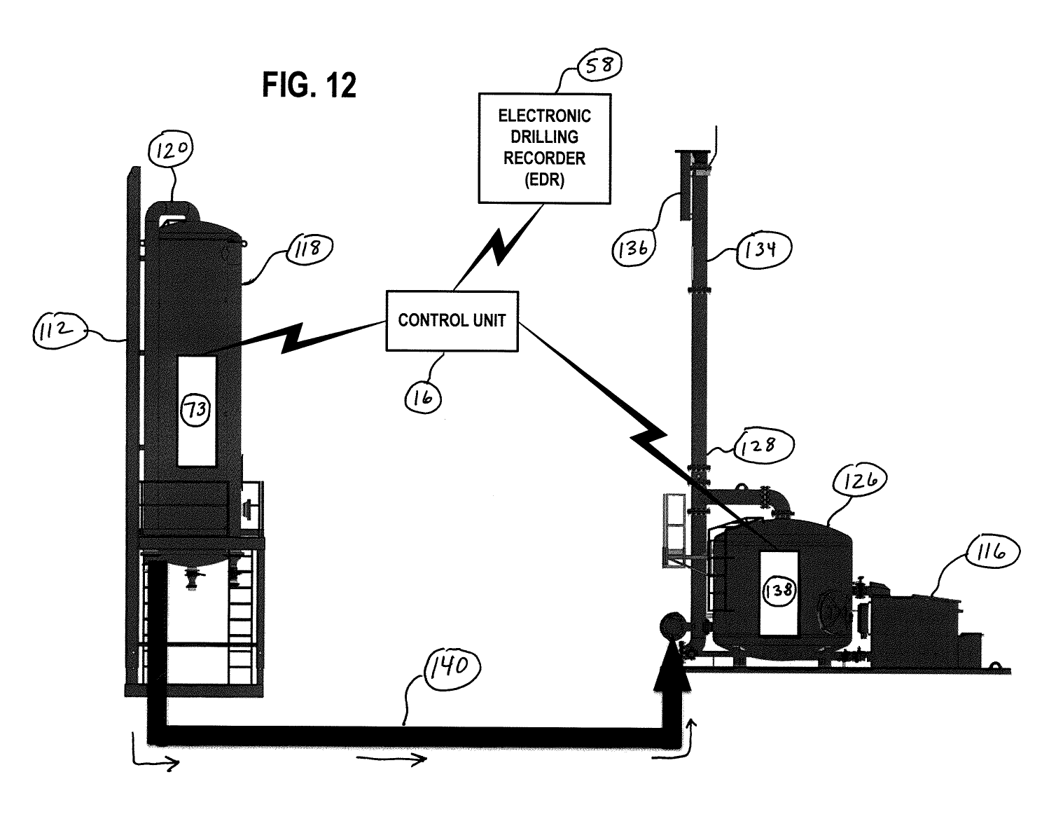

In an exemplary embodiment, as illustrated in FIG. 12 with continuing reference to FIGS. 1-11, a flow path is generally referred to by the reference numeral 140. The flow path 140 represents the flow of materials, from the mud-gas separator vessel 118 of the mud-gas containment system 112 and to the mud-gas containment vessel 126 of the mud-gas containment system 116, via at least the gas vent line 120, the joint 130, and the gas vent line 132. In several exemplary embodiments, the sensor housing assembly 73 is used to provide an early warning of potential flooding within the mud-gas separator vessel 118, providing an even earlier warning of potential flooding within the mud-gas containment vessel 126. In several exemplary embodiments, the simultaneous monitoring of the mud-gas separator vessel 118 and the mud-gas containment vessel 126 provides the opportunity to respond much earlier to fluid level changes.

In several exemplary embodiments, the simultaneous monitoring of the mud-gas separator vessel 118, the shale-gas separator vessel 122, the gas vent line 132, and the mud-gas containment vessel 126 provides the opportunity to respond much earlier to fluid level changes.

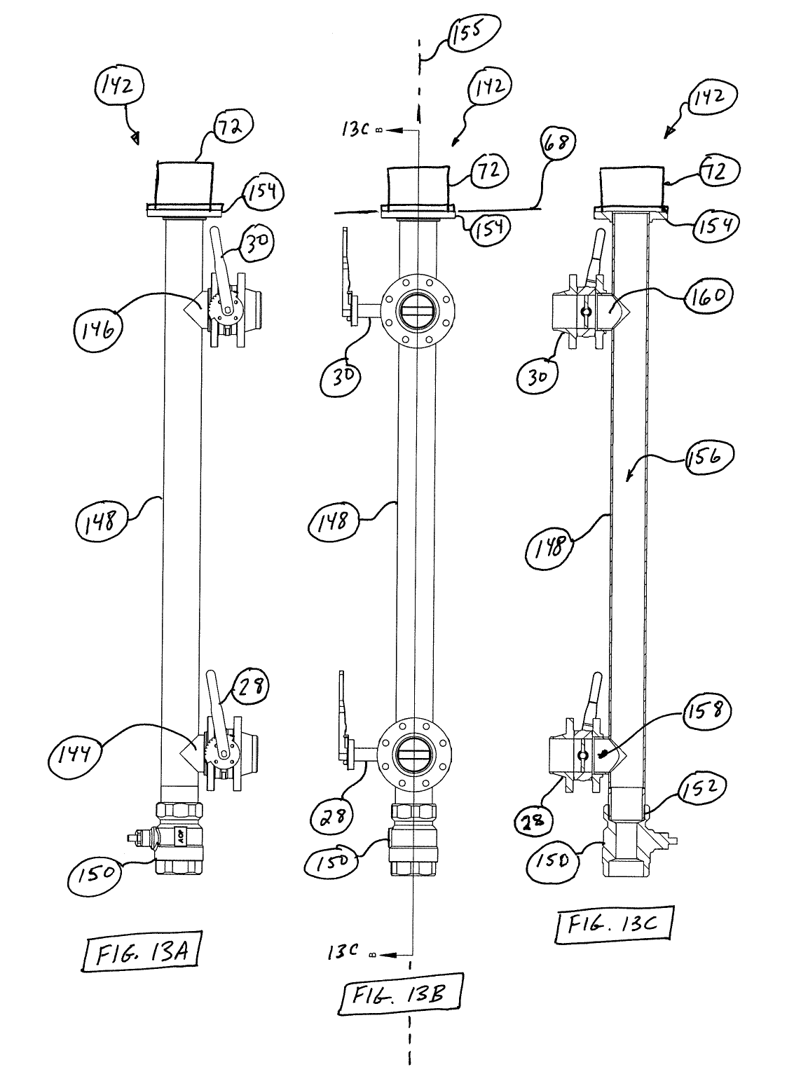

In an exemplary embodiment, as illustrated in FIGS. 13A, 13B, and 13C with continuing reference to FIGS. 1-12, still yet another exemplary embodiment of the sensor housing assembly 12 of FIG. 1 is generally referred to by the reference numeral 142. The sensor housing assembly 142 includes fittings 144 and 146, and a tubular housing 148 extending therebetween (the tubular housing 148 also extends beyond each of the fittings 144 and 146). The isolation valves 28 and 30 are connected to the fittings 144 and 146, respectively. A drain plug 150 is connected to the tubular housing 148 at the lower end thereof; in an exemplary embodiment, the tubular housing 148 includes an external threaded connection 152 at its lower end, and the drain plug 150 is threadably engaged with the external threaded connection to connect the drain plug 150 to the tubular housing 148. A flange 154 is directly connected to the upper end of the tubular housing 148, leaving the top end of the tubular housing 148 open, thereby defining a port. The level sensor 72 is connected to the tubular housing 148 via at least the flange 154 so that at least a portion of the level sensor 72 is adjacent the open end of the tubular housing 148 (the port). In an exemplary embodiment, the level sensor 72 is a non-contact radar level sensor. The tubular housing 148 defines a longitudinally-extending center axis 155, which is perpendicular to the open end of the tubular housing 148 (the port). In several exemplary embodiments, the cap 66 (not shown) is connected to the flange 154. The cap 66 lies in the plane 68, which is perpendicular to the longitudinally-extending center axis 155. The port 70 (not shown) is formed through the cap 66, and is in fluid communication with an internal region 156 defined by the tubular housing 148; at least a portion of the level sensor 72 is adjacent the port 70. The fittings 144 and 146 define internal passages 158 and 160, respectively.

As shown in FIGS. 13A, 13B, and 13C, the fittings 144 and 146 are connected directly to the tubular housing 148. In an exemplary embodiment, the fittings 144 and 146 are connected directly to the tubular housing 148 using saddle welds. In an exemplary embodiment, the fittings 144 and 146 are connected directly to the tubular housing 148 so that the respective direct connections between the tubular housing 148 and each of the fittings 144 and 146 are weld-less, within the internal region 156 defined by the tubular housing 148, increasing smoothness along respective internal surfaces of the tubular housing 148 and the fittings 144 and 146.

In operation, in an exemplary embodiment, the sensor housing assembly 142 is part of the intelligent sensor system 10 of FIG. 1 and operates in a manner substantially identical to the manner in which the intelligent sensor system 10 of FIG. 1 operates with the sensor housing assembly 60 of FIGS. 5 and 6.

During operation, the perpendicular orientation between the center axis 155, and the port to which at least a portion of the level sensor 72 is adjacent, facilitates the measurement of the fluid level 50 by the level sensor 72.

During operation, in several exemplary embodiments, the level sensor 72 is a non-contact radar level sensor, and the respective direct connections between the tubular housing 148 and each of the fittings 144 and 146, which are weld-less within the internal region 156, increase smoothness along respective internal surfaces of the tubular housing 148 and the fittings 144 and 146, thereby also facilitating the measurement of the fluid level 50 by the non-contact radar level sensor.

In several exemplary embodiments, the ports 74, 76, and 78 may be formed in the wall of the tubular housing 148, and the pressure sensors 80 and 82 may be connected to the tubular housing 148 at the ports 74 and 76, respectively. With these modifications, in operation, in an exemplary embodiment, the sensor housing assembly 142 is part of the intelligent sensor system 10 of FIG. 1 and operates in a manner substantially identical to the manner in which the intelligent sensor system 10 of FIG. 1 operates with the sensor housing assembly 73 of FIGS. 7A and 7B.

In several exemplary embodiments, the ports 74, 76, and 78 may be formed in the wall of the tubular housing 148, and the pressure sensors 80 and 82 may be connected to the tubular housing 148 at the ports 74 and 76, respectively. Moreover, the level sensor 72 may be removed and instead the solid cap 94 may be connected to the flange 154. With these modifications, in operation, in an exemplary embodiment, the sensor housing assembly 142 is part of the intelligent sensor system 10 of FIG. 1 and operates in a manner substantially identical to the manner in which the intelligent sensor system 10 of FIG. 1 operates with the sensor housing assembly 92 of FIG. 9A.

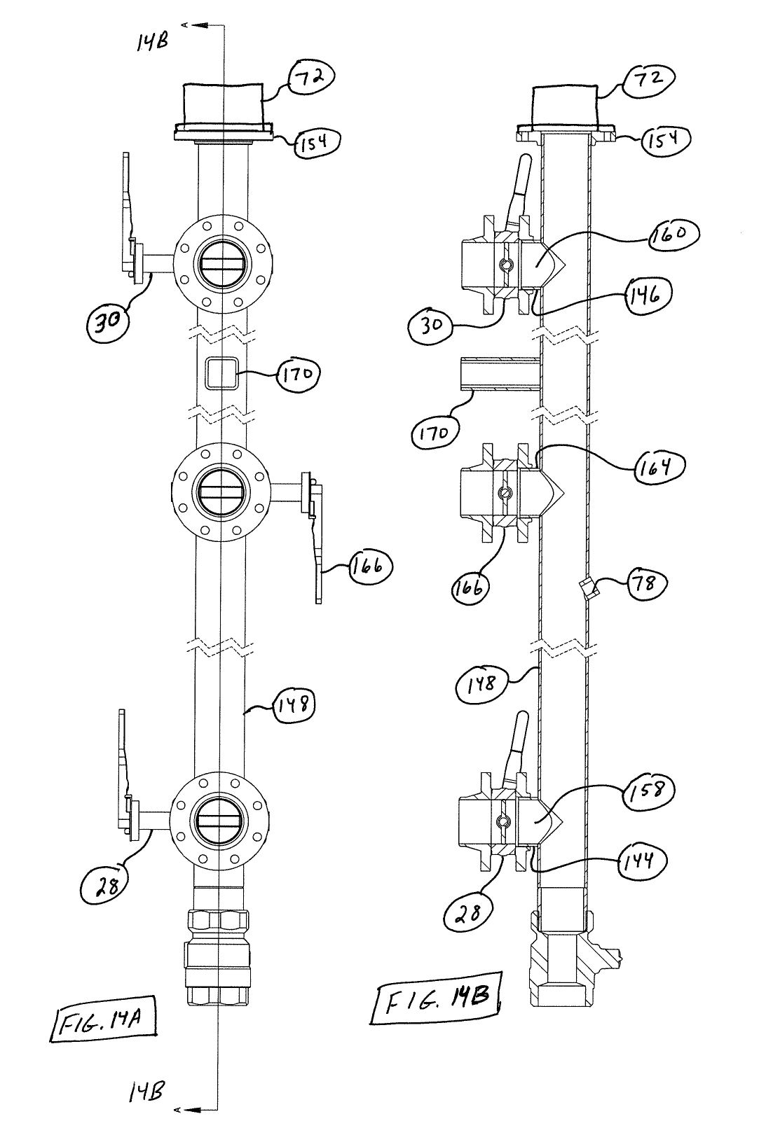

In an exemplary embodiment, as illustrated in FIGS. 14A and 14B with continuing reference to FIGS. 1-13C, still yet another exemplary embodiment of the sensor housing assembly 12 of FIG. 1 is generally referred to by the reference numeral 162. The sensor housing assembly 162 includes all of the components of the sensor housing assembly 142, which identical components are given the same reference numerals. The sensor housing assembly 162 further includes a fitting 164, which is connected directly to the tubular housing 148 and vertically positioned between the fittings 144 and 146. A valve 166 is connected to the fitting 164. The fitting 164 defines an internal passage 168. A protrusion 170 extends from the tubular housing 148. The water jet port 78 is shown in FIG. 14B. In several exemplary embodiments, the tubular housing 148 of the sensor housing assembly 162 is longer than the tubular housing 148 of the sensor housing assembly 142.

The operation of the sensor housing assembly 162 is substantially similar to the above-described operation of the sensor housing assembly 142. The above-described modifications to the sensor housing assembly 142, and the corresponding operations, are equally applicable to the sensor housing 162.

As shown in FIGS. 14A and 14B, the fittings 144, 146, and 164 are connected directly to the tubular housing 148. In an exemplary embodiment, the fittings 144, 146, and 164 are connected directly to the tubular housing 148 using saddle welds. In an exemplary embodiment, the fittings 144, 146, and 164 are connected directly to the tubular housing 148 so that the respective direct connections between the tubular housing 148 and each of the fittings 144, 146, and 164 are weld-less, within the internal region 156 defined by the tubular housing 148, increasing smoothness along respective internal surfaces of the tubular housing 148 and the fittings 144, 146, and 164. This increased smoothness facilitates the operation of the level sensor 72, especially when the level sensor 72 is a non-contact radar level sensor.

In several exemplary embodiments, a plurality of instructions, or computer program(s), are stored on a non-transitory computer readable medium, the instructions or computer program(s) being accessible to, and executable by, one or more processors. In several exemplary embodiments, the one or more processors execute the plurality of instructions (or computer program(s)) to operate in whole or in part the above-described exemplary embodiments. In several exemplary embodiments, the one or more processors are part of the control unit 16, the EDR 58, one or more other computing devices, or any combination thereof. In several exemplary embodiments, the non-transitory computer readable medium is part of the control unit 16, the EDR 58, one or more other computing devices, or any combination thereof.

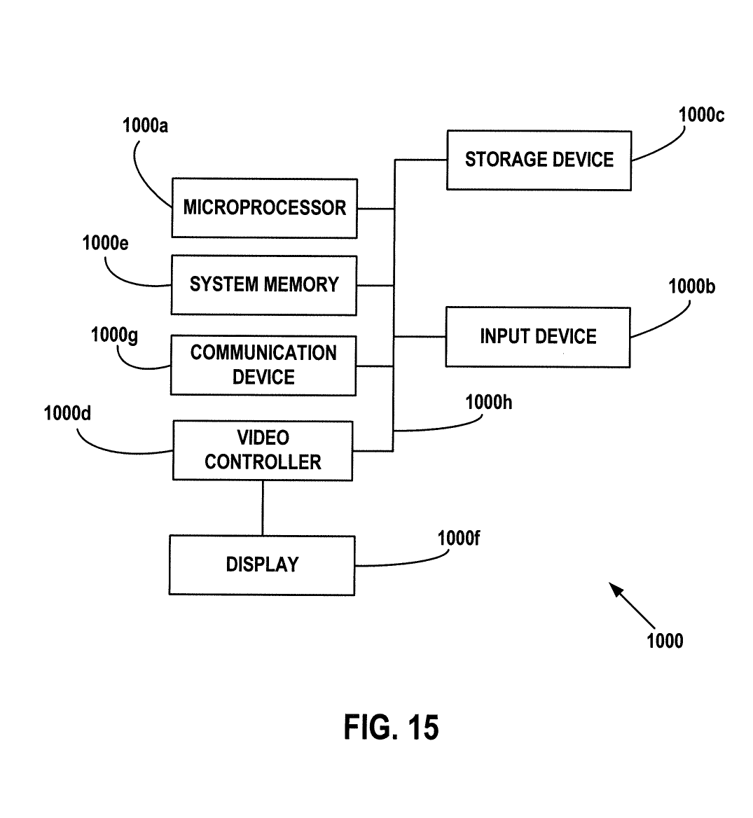

In an exemplary embodiment, as illustrated in FIG. 15 with continuing reference to FIGS. 1-24, an illustrative computing device 1000 for implementing one or more embodiments of one or more of the above-described networks, elements, methods and/or steps, and/or any combination thereof, is depicted. The computing device 1000 includes a microprocessor 1000a, an input device 1000b, a storage device 1000c, a video controller 1000d, a system memory 1000e, a display 1000f, and a communication device 1000g all interconnected by one or more buses 1000h. In several exemplary embodiments, the storage device 1000c may include a floppy drive, hard drive, CD-ROM, optical drive, any other form of storage device and/or any combination thereof. In several exemplary embodiments, the storage device 1000c may include, and/or be capable of receiving, a floppy disk, CD-ROM, DVD-ROM, or any other form of computer-readable medium that may contain executable instructions. In several exemplary embodiments, the communication device 1000g may include a modem, network card, or any other device to enable the computing device to communicate with other computing devices. In several exemplary embodiments, any computing device represents a plurality of interconnected (whether by intranet or Internet) computer systems, including without limitation, personal computers, mainframes, PDAs, smartphones and cell phones.

In several exemplary embodiments, one or more of the components of the above-described exemplary embodiments include at least the computing device 1000 and/or components thereof, and/or one or more computing devices that are substantially similar to the computing device 1000 and/or components thereof. In several exemplary embodiments, one or more of the above-described components of the computing device 1000 include respective pluralities of same components.

In several exemplary embodiments, a computer system typically includes at least hardware capable of executing machine readable instructions, as well as the software for executing acts (typically machine-readable instructions) that produce a desired result. In several exemplary embodiments, a computer system may include hybrids of hardware and software, as well as computer sub-systems.

In several exemplary embodiments, hardware generally includes at least processor-capable platforms, such as client-machines (also known as personal computers or servers), and hand-held processing devices (such as smart phones, tablet computers, personal digital assistants (PDAs), or personal computing devices (PCDs), for example). In several exemplary embodiments, hardware may include any physical device that is capable of storing machine-readable instructions, such as memory or other data storage devices. In several exemplary embodiments, other forms of hardware include hardware sub-systems, including transfer devices such as modems, modem cards, ports, and port cards, for example.

In several exemplary embodiments, software includes any machine code stored in any memory medium, such as RAM or ROM, and machine code stored on other devices (such as floppy disks, flash memory, or a CD ROM, for example). In several exemplary embodiments, software may include source or object code. In several exemplary embodiments, software encompasses any set of instructions capable of being executed on a computing device such as, for example, on a client machine or server.

In several exemplary embodiments, combinations of software and hardware could also be used for providing enhanced functionality and performance for certain embodiments of the present disclosure. In an exemplary embodiment, software functions may be directly manufactured into a silicon chip. Accordingly, it should be understood that combinations of hardware and software are also included within the definition of a computer system and are thus envisioned by the present disclosure as possible equivalent structures and equivalent methods.

In several exemplary embodiments, computer readable mediums include, for example, passive data storage, such as a random access memory (RAM) as well as semi-permanent data storage such as a compact disk read only memory (CD-ROM). One or more exemplary embodiments of the present disclosure may be embodied in the RAM of a computer to transform a standard computer into a new specific computing machine. In several exemplary embodiments, data structures are defined organizations of data that may enable an embodiment of the present disclosure. In an exemplary embodiment, a data structure may provide an organization of data, or an organization of executable code.

In several exemplary embodiments, any networks and/or one or more portions thereof, may be designed to work on any specific architecture. In an exemplary embodiment, one or more portions of any networks may be executed on a single computer, local area networks, client-server networks, wide area networks, internets, hand-held and other portable and wireless devices and networks.

In several exemplary embodiments, a database may be any standard or proprietary database software. In several exemplary embodiments, the database may have fields, records, data, and other database elements that may be associated through database specific software. In several exemplary embodiments, data may be mapped. In several exemplary embodiments, mapping is the process of associating one data entry with another data entry. In an exemplary embodiment, the data contained in the location of a character file can be mapped to a field in a second table. In several exemplary embodiments, the physical location of the database is not limiting, and the database may be distributed. In an exemplary embodiment, the database may exist remotely from the server, and run on a separate platform. In an exemplary embodiment, the database may be accessible across the Internet. In several exemplary embodiments, more than one database may be implemented.

In several exemplary embodiments, a plurality of instructions stored on a non-transitory computer readable medium may be executed by one or more processors to cause the one or more processors to carry out or implement in whole or in part the above-described operation of each of the above-described exemplary embodiments of the intelligent sensor system 10, the system 110, the method 84, the method 96, and/or any combination thereof. In several exemplary embodiments, such a processor may include one or more of the microprocessor 1000a, the processor 32, and/or any combination thereof, and such a non-transitory computer readable medium may include the computer readable medium 34 and/or may be distributed among one or more components of the intelligent sensor system 10 and/or the system 110. In several exemplary embodiments, such a processor may execute the plurality of instructions in connection with a virtual computer system. In several exemplary embodiments, such a plurality of instructions may communicate directly with the one or more processors, and/or may interact with one or more operating systems, middleware, firmware, other applications, and/or any combination thereof, to cause the one or more processors to execute the instructions.

In the foregoing description of certain embodiments, specific terminology has been resorted to for the sake of clarity. However, the disclosure is not intended to be limited to the specific terms so selected, and it is to be understood that each specific term includes other technical equivalents which operate in a similar manner to accomplish a similar technical purpose. Terms such as “left” and right”, “front” and “rear”, “above” and “below” and the like are used as words of convenience to provide reference points and are not to be construed as limiting terms.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

In addition, the foregoing describes only some embodiments of the invention(s), and alterations, modifications, additions and/or changes can be made thereto without departing from the scope and spirit of the disclosed embodiments, the embodiments being illustrative and not restrictive.

Furthermore, invention(s) have described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention(s). Also, the various embodiments described above may be implemented in conjunction with other embodiments, e.g., aspects of one embodiment may be combined with aspects of another embodiment to realize yet other embodiments. Further, each independent feature or component of any given assembly may constitute an additional embodiment.

What is claimed is:

1. A system adapted to monitor at least a first operating parameter of a first vessel, the first vessel defining a first internal region, the system comprising:
a first sensor housing assembly, the first sensor housing assembly comprising:
a first fitting adapted to be connected to the first vessel, the first fitting defining a first internal passage adapted to be in fluid communication with the first internal region;
a second fitting adapted to be connected to the first vessel, the second fitting defining a second internal passage adapted to be in fluid communication with the first internal region;
a housing extending between the first and second fittings, the housing defining a second internal region adapted to be in fluid communication with the first internal region via the first and second passages; and
a first sensor connected to at least one of the first fitting, the second fitting, and the housing;
wherein the first sensor is adapted to measure a first physical property associated with the first vessel; and
wherein the monitored first operating parameter is, or is based on, the first physical property measured by the first sensor.

2. The system of claim 1, further comprising:
a control unit adapted to be in communication with the first sensor and adapted to receive from the first sensor first measurement data associated with the first physical property;
wherein the control unit is adapted to determine the first operating parameter based on the first measurement data.

3. The system of claim 2, wherein the control unit is adapted to be in communication with an electronic drilling recorder (EDR);
wherein the control unit is adapted to send to the EDR first parameter data associated with first operating parameter;
wherein the housing is a tubular housing, the tubular housing comprising opposing first and second end portions; and
wherein the system further comprises:
a first t-fitting connected to the first end portion of the tubular housing, wherein the first fitting is part of the first t-fitting; and
a second t-fitting connected to the second end portion of the tubular housing, wherein the second fitting is part of the second t-fitting.

4. The system of claim 1, wherein the first physical property is a fluid level within the first vessel;
wherein the first sensor is a level sensor adapted to measure the fluid level within the first vessel;
wherein the level sensor is one of a guided wave level sensor and a non-contact radar level sensor;
wherein the first sensor housing assembly further comprises a port in fluid communication with the second internal region of the housing;
wherein the level sensor is positioned, relative to the port, so that the level sensor can measure the fluid level within the first vessel;
wherein the housing defines a longitudinally-extending center axis;
wherein the first housing assembly further comprises a cap lying in a plane that is perpendicular to the center axis of the housing;
wherein the first port is formed through the cap and the level sensor is connected to the cap; and
wherein the perpendicular orientation between the center axis and the plane in which the cap lies facilitates the measurement of the fluid level by the level sensor.

5. The system of claim 4, wherein the level sensor is the non-contact radar level sensor, at least a portion of which is positioned adjacent the first port;
wherein the housing is a tubular housing;
wherein each of the first and second fittings is connected directly to the tubular housing; and
wherein the respective direct connections between the tubular housing and each of the first and second fitting are weld-less, within the second internal region defined by the tubular housing, increasing smoothness along respective internal surfaces of the tubular housing and the first and second fittings, facilitates the measurement of the fluid level by the non-contact radar level sensor.

6. The system of claim 1, wherein the first sensor housing assembly further comprises a second sensor connected to at least one of the first fitting, the second fitting, and the housing;
wherein the second sensor is adapted to measure a second physical property associated with the first vessel;
wherein the first sensor housing assembly further comprises:
a first end portion at which the first fitting is located;
a second end portion at which the second fitting is located, the second end portion opposing the first end portion;
a first port formed at the first end portion of the first sensor housing assembly, wherein the first port is in fluid communication with the second internal region of the housing;
and
a second port formed at the second end portion of the first sensor housing assembly, wherein the second port is in fluid communication with the second internal region of the housing;
wherein the first and second sensors are first and second pressure sensors, respectively; and
wherein the first and second pressure sensors are positioned adjacent the first and second ports, respectively.

7. The system of claim 6, wherein the first physical property adapted to be measured by the first pressure sensor is mud column pressure within the first vessel;
wherein the second physical property adapted to be measured by the second pressure sensor is gas vessel pressure within the first vessel; and
wherein the monitored first operating parameter is one of:
mud density; and
mud discharge flow rate, the mud discharge flow rate being based on at least the mud column pressure and operating characteristics of a discharge valve via which mud is adapted to be discharged from the first vessel.

8. The system of claim 6, wherein the first physical property to be measured by the first pressure sensor is pressure at a lower end portion of the first vessel;
wherein the second physical property to be measured by the second pressure sensor is pressure at the upper end portion of the first vessel; and
wherein the monitored first operating parameter is selected from the group consisting of a fluid level within the first vessel; an operating pressure within the first vessel;
and liquid density within the first vessel.

9. The system of claim 1, further comprising:
a second sensor housing assembly, the second sensor housing assembly comprising a second sensor adapted to measure a second physical property associated with a second vessel; and
a control unit adapted to be in communication with each of the first and second sensors;
wherein the control unit is adapted to receive from the first sensor first measurement data associated with the first physical property;
wherein the control unit is adapted to receive from the second sensor second measurement data associated with the second physical property;
wherein the control unit is adapted to determine the first operating parameter based on the first measurement data;
wherein the control unit is adapted to determine a second operating parameter of the second vessel based on the second measurement data; and
wherein the second operating parameter is, or is based on, the second physical property measured by the second sensor.

10. The system of claim 9, further comprising:
the first vessel, wherein the first vessel is a mud-gas separator vessel located at a drilling rig site;
the second vessel, wherein the second vessel is a mud-gas containment vessel located located at the drilling rig site; and
a gas vent line via which the mud-gas containment vessel is in fluid communication with the mud-gas separator vessel;
wherein the first sensor housing assembly is connected to the mud-gas separator vessel;
wherein the second sensor housing assembly is connected to the mud-gas containment vessel;
wherein the first and second sensors are level sensors adapted to measure respective fluid levels within the mud-gas separator vessel and the mud-gas containment vessel; and
wherein the monitored first operating parameter of the mud-gas separator vessel provides an early warning of potential flooding within the mud-gas separator vessel and an even earlier warning of potential flooding within the mud-gas containment vessel.

11. A monitoring system located at a drilling rig site, the system comprising:
a first vessel;
a second vessel in fluid communication with the first vessel;
a first sensor housing assembly connected to the first vessel, the first sensor housing comprising a first sensor adapted to measure a first physical property associated with the first vessel;
a second sensor housing assembly connected to the second vessel, the second sensor housing comprising a second sensor adapted to measure a second physical property associated with the second vessel; and
a control unit adapted to be in communication with each of the first and second sensors to determine and monitor first and second operating parameters of the first and second vessels, respectively;
wherein each of the first and second operating parameters is, or is based on, the first and second physical properties, respectively.

12. The system of claim 11, further comprising an electronic drilling recorder (EDR) in communication with the control unit;
wherein the control unit is adapted to send to the EDR parameter data associated with first and second operating parameters; and
wherein each of the first and second sensors is one of the following:
a level sensor adapted to measure a fluid level within the first or second vessel; and
a pressure sensor adapted to measure pressure within the first or second vessel.

13. The system of claim 11, wherein the first vessel is a mud-gas separator vessel;
wherein the second vessel is a mud-gas containment vessel;
wherein the first sensor housing assembly is connected to the mud-gas separator vessel;
wherein the second sensor housing assembly is connected to the mud-gas containment vessel;
wherein the first and second sensors are level sensors adapted to measure respective fluid levels within the mud-gas separator vessel and the mud-gas containment vessel; and
wherein the monitored first operating parameter of the mud-gas separator vessel provides an early warning of potential flooding within the mud-gas separator vessel and an even earlier warning of potential flooding within the mud-gas containment vessel.

14. The system of claim 11, further comprising a discharge valve via which mud is adapted to flow out of one of the first and second vessels;
wherein the control unit controls the discharge valve based on at least one of the first and second operating parameters; and
wherein each of the first and second vessels is selected from the group consisting of: a mud-gas separator vessel; a shale-gas separator vessel; and a mud-gas containment vessel.

15. The system of claim 11, further comprising:
a gas vent line via which the second vessel is in fluid communication with the first vessel; and
a third sensor housing assembly connected to the gas vent line, the third sensor housing assembly comprising a third sensor adapted to measure a third physical property associated with the second vessel;
wherein the control unit is in communication with the third sensor to determine and monitor a third operating parameter of the gas vent line; and
wherein the third operating parameter is, or is based on, the third physical property;
wherein the third operating parameter is selected from the group consisting of: existence of hydrocarbons within the gas vent line; flammables content within the gas vent line; and gas flow rate within the gas vent line; and
wherein the system further comprises a flare stack in fluid communication with the gas vent line, the flare stack comprising an igniter; and
wherein the control unit controls the operation of the igniter based on the third operating parameter of the gas vent line.

16. A system adapted to monitor at least a first operating parameter of a gas vent line, the system comprising:
a sensor housing assembly adapted to be connected to the gas vent line, the sensor housing assembly comprising a first sensor adapted to measure a first physical property associated with the gas vent line;
wherein the monitored first operating parameter is, or is based on, the first physical property measured by the first sensor.

17. The system of claim 16, wherein the third operating parameter is selected from the group consisting of: existence of hydrocarbons within the gas vent line; flammables content within the gas vent line; and gas flow rate within the gas vent line.

18. The system of claim 16, further comprising:
a control unit adapted to be in communication with the first sensor and adapted to receive from the first sensor first measurement data associated with the first physical property;
wherein the control unit is adapted to determine the first operating parameter based on the first measurement data.

19. The system of claim 18, wherein the control unit is adapted to control the operation of an igniter of a flare stack, the flare stack being in fluid communication with the gas vent line;
wherein the control unit controls the operation of the igniter based on the first operating parameter of the gas vent line.

20. The system of claim 18, wherein the control unit is adapted to be in communication with an electronic drilling recorder (EDR);
wherein the control unit is adapted to send to the EDR first parameter data associated with first operating parameter;
wherein the sensor housing assembly further comprises:
a first fitting adapted to be connected to the gas vent line, the first fitting defining a first internal passage adapted to be in fluid communication with the gas vent line;
a second fitting adapted to be connected to the first vessel, the second fitting defining a second internal passage adapted to be in fluid communication with the gas vent line; and
a housing extending between the first and second fittings, the housing defining a second internal region adapted to be in fluid communication with the gas vent line;
and
wherein the first sensor is connected to at least one of the first fitting, the second fitting, and the housing.
